(19) United States
(12) Patent Application Publication
(10) Pub. No.: US 2026/0325499 A1
(43) Pub. Date: Oct. 22, 2026

(54) S1P1 receptor modulators for use in the treatment of type 1 IFN mediated diseases

(71) Applicant: VIATRIS ASIA PACIFIC PTE. LTD., Singapore (SG)

(72) Inventors: Daniel STRASSER, Allschwil (CH); Mark MURPHY, Allschwil (CH)

(21) Appl. No.: 19/111,374

(22) PCT Filed: Sep. 12, 2023
(86) PCT No.: PCT/EP2023/074961

§ 371 Date: Mar. 13, 2025

Related U.S. Application Data
(60) Provisional application No. 63/375,668, filed on Sep. 14, 2022.

(51) Int. Cl. A61K 31/4439; C12Q 1/6883

(52) U.S. Cl. CPC A61K 31/4439; C12Q 1/6883

(57) ABSTRACT

The present invention relates to a a S1P₁ receptor modulator, or a pharmaceutically acceptable salt thereof, for use in the prevention or treatment of a type 1 interferon mediated disease in a subject that has a high type 1 interferon (IFN-1) gene signature score or elevated levels of interferon-alpha (IFN-α) and/or interferon-gamma (IFN-γ) in blood plasma.

### FIELD OF THE INVENTION

The present invention relates to a S1P₁ receptor modulator, or a pharmaceutically acceptable salt thereof, for use in the prevention or treatment of a type 1 interferon mediated disease in a subject that has a high type 1 interferon (IFN-1) gene signature score or elevated levels of interferon-alpha (IFN-α) and/or interferon-gamma (IFN-γ) in blood plasma.

### BACKGROUND OF THE INVENTION

Sphingosine 1-phosphate (S1P) is a lipid mediator formed by the metabolism of sphingomyelin. In vertebrates, S1P is secreted into the extracellular environment and signals via G protein coupled S1P receptors including the S1P1 receptor which plays an important role in lymphocyte trafficking [Mendelson, K. et al., Development 2014, 141, 5-9, PMID:24346695].

S1P1 receptor modulators are approved for the treatment of multiple sclerosis and Inflammatory Bowel Disease and are being investigated in further inflammatory diseases involving lymphocyte driven inflammatory processes [Burg, N. et al., Nat Rev Rheumatol. 2022, 18(6), 335-351, PMID:35508810].

(S)-3-{4-[5-(2-cyclopentyl-6-methoxy-pyridin-4-yl)-[1,2,4]oxadiazol-3-yl]-2-ethyl-6-methyl-phenoxy}-propane-1,2-diol (hereinafter also referred to as “COMPOUND” or “cenerimod”) is a potent, selective, and orally active S1P1 receptor modulator which prevents the egress of lymphocytes from secondary lymphoid organs into the vascular circulation, via internalization of the S1P1 receptor. As such, its immunomodulatory role in autoimmune diseases may be of benefit in treating such diseases in which lymphocytes play an important role in propagating disease. The preparation of (S)-3-{4-[5-(2-cyclopentyl-6-methoxy-pyridin-4-yl)-[1,2,4]oxadiazol-3-yl]-2-ethyl-6-methyl-phenoxy}-propane-1,2-diol and the medicinal uses thereof are described in PCT applications WO 2011/007324, WO 2013/175397 and WO 2016/184939.

Both B and T lymphocytes have been implicated in playing a major role in the pathogenesis of systemic lupus erythematosus (SLE), a complex and heterogeneous autoimmune disease of unknown etiology, characterized by the production of high titers of pathogenic autoantibodies, infiltrating lymphocytes, and tissue damage across multiple organ systems [Rahman, A. et al., N Engl J Med 2008, 358, 929-939, PMID:18305268].

In a phase 2 clinical trial cenerimod reduced both B and T lymphocytes in the circulation confirming the mode of action (Hermann V, et al. Lupus Science & Medicine 2019; 6:e000354. doi:10.1136/lupus-2019-000354). Furthermore, cenerimod reduced interferon (IFN)-associated proteins in plasma [Strasser, D S et al., RMD Open 2020, 6, e001261, PMID:32917831].

IFNs are central effector molecules produced in large amounts during an immune response to infection, activating defense mechanisms and resulting in the inhibition of microbial replication. Type 1 IFNs are the largest group and comprise several classes and subclasses. Almost all cells can produce IFN-α and IFN-β, members of the Type 1 IFNs, including dendritic-, monocytic-T and B cells of the immune system. IFN-γ is the only type 2 IFN and is mainly produced by NK cells and T cells of the immune system [McNab, F. et al., Nat Rev Immunol 2015, 15, 87-103, PMID:25614319; Rönnblom, L. et al., Lupus Sci Med 2019, 6, e000270, PMID:31497305; Colonna, M. et al., Current Opinion Immunol 2002, 14, 373-379, PMID:11973137].

Type-1 IFNs signal through a heterodimeric transmembrane receptor composed of the subunits IFNAR1 and IFNAR2, broadly expressed across multiple cell types. Similarly, type 2 IFN engage with the heterodimer IFNGR1 and IFNGR2. Both IFNAR and IFNGR complexes recruit JAK kinase family members. IFNAR recruits TYK2 and JAK1 while IFNGR recruits JAK1 and JAK2. This leads to subsequent STAT1 and STAT2 phosphorylation and the subsequent translocation of the signaling pathway transcription factors into the nucleus. These in turn bind to IFN response elements on the DNA, initiating the transcription of a whole host of genes [Platanias, L C., Nat Rev Immunol 2005, 5, 375-386, PMID:15864272].

Importantly, the activation of these pathways is to a certain degree overlapping. The activation of the gene transcription by IFNs can be described with an IFN gene signature, can consist of many hundreds of genes, is dependent on the kinetics and cell types involved, and can lead to a multitude of outcomes and nuances [Rönnblom, L. et al., Curr Opin Rheumatol 2013, 25(2), 248-253, PMID:23249830].

The observation that IFNs are elevated in SLE was already made in the late 1970s and an indication that IFNs may in fact cause SLE-like symptoms comes from studies of interferonopathies where a constitutive overproduction of Type 1 IFNs caused by genetic mutations in genes within both the type 1 and type 2 IFN inducing pathway have been found. These individuals have a prominent IFN signature, and some patients present an SLE like-phenotype [Lee-Kirsch, M A., Annu Rev Med 2017, 68, 297-315, PMID:27813875]. The reasons for elevated IFN levels in SLE are not clearly understood and not all patients diagnosed with SLE have an elevated type 1 IFN [Northcott, M. et al., Lupus Sci Med 2022, 9, e000625, PMID:35197305]. There are many lines of evidence suggesting genetic predisposition and environmental risk factors may lead to elevated levels of IFNs. Principally this reflects the involvement of multiple pattern recognition receptors in IFN activation mentioned above and their means of activation. A prominent mechanism is thought to be immune complexes which consist of autoantibodies, for example anti-dsDNA and anti-nuclear antibodies, nucleic acids, and nucleic acid binding proteins. This suggests nucleic acids as potential trigger and having autoantibodies against nucleic acids represents a risk factor to develop SLE.

Nucleic acids are sensed among other cells by plasmacytoid dendritic cells (pDCs), which are thought to be a key cell type producing type 1 IFNs in SLE.

Increased IFN signaling has not only been demonstrated in SLE but in other autoimmune diseases too, although the frequency and distribution within the population is unique to each autoimmune disease [Rönnblom, L. et al., Curr Opin Rheumatol 2013, 25(2), 248-253, PMID:23249830].

Although pDCs produce large amounts of IFN Type 1 [Cella, M. et al., Nature Med 1999, 5(8), 919-923, PMID:10426316], other cell types may be involved in IFN production in SLE including keratinocytes, endothelial cells, fibroblasts and monocytes, NK cells, B cells and T cells. Finally, genetic risk factors have been identified in SLE that are associated with IFN production or responses to IFNs further supporting the hypothesis that perturbations in IFN regulation may contribute to SLE [Deng, Y. et al., Curr Rheumatol Rep 2017, 19, 68 PMID:28983873, Catalina, M D. et al., Communications Biology 2019, 2(140), https://doi.org/10.1038/s42003-019-0382-x, PMID:31044165].

Gene transcription analysis of IFN-induced genes was initially performed in 2003 utilizing peripheral blood from SLE patients which demonstrated that hundreds of genes associated with IFN activation were induced [Baechler, E C. et al., PNAS 2003, 100(5), 2610-2615, PMID:12604793] and provided evidence of an IFN gene signature in patients with SLE. Therefore, the IFN-1 signature reflects IFN-regulated genes, meaning that those genes are upregulated due to the biological activity of type 1 IFNs. Subsequent studies have demonstrated between 50 and 80% of SLE patients show a consistent presence of an IFN-1 signature from the blood and this is stable over time [Northcott, M. et al., Lupus Sci Med 2022, 9, e000625, PMID:35197305; Kirou, K A. et al., Arthritis Rheumatism, 2004, 50(12), 3958-3967, PMID:15593221; Oke, V. et al., Arthritis Research Therapy 2019, 21, 107, PMID:31036046; Psarras, A. et al., Rheumatology 2017, 56, 1662-1675, PMID:28122959; Rönnblom, L. et al., Lupus Sci Med 2019, 6, e000270, PMID:31497305]. The communality between all IFN-1 gene signatures lies in the fact that IFN response elements are present in the genes induced by IFN signaling.

An increase in IFN signaling has broad and wide-ranging effects on the body and increased IFN production may be linked to potential disease manifestations in patients with SLE, the principal ones are summarized below.

A prominent association is seen between elevated IFN and SLE associated nephritis [Feng, X. et al., Arthritis Rheumatism 2006, 54(9), 2951-2962, PMID:16947629; Kirou, K A. et al., Arthritis Rheumatism, 2005, 52(5), 1491-1503, PMID:15880830]. Further, IFN has also been shown to be high in the kidney tissue suggesting a strong link of IFN with kidney damage [Castellano, G. et al., Arthritis Research Therapy 2015, 17, 72, PMID:25889472].

This evidence clearly suggests that IFNs participate in SLE disease, however the association of a blood IFN signature and clinical disease characteristics is not limited to any one manifestation. Further the IFN signature does not distinguish between high and low disease activity [Kennedy, W P. et al., Lupus Sci Med 2015, 2, e000080, PMID:25861459], as defined by various criteria, but can predict the subsequent disease severity over time [Mai, L. et al., Arthritis Research Therapy 2021, 23, 29, PMID:33451338].

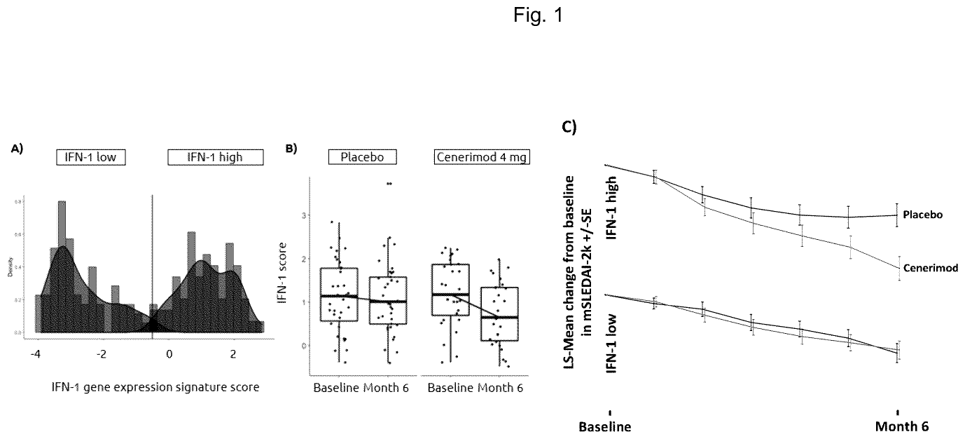

Data on the distribution of type 1 IFN gene signature high vs IFN signature low within the general SLE population is somewhat limited but reports have placed it in the range of 60-80% [Psarras, A. et al., Rheumatology 2017, 56, 1662-1675, PMID:28122959]. An important property of the IFN-1 gene signature is the bimodal distribution in patients with SLE allowing separation into high or low IFN-1 gene signature strata [El-Sherbiny, Y M. et al., Scientific Reports 2018, 8, 5793, DOI:10.1038/s41598-018-24198-1, PMID:29643425] and observed in a double-blind, randomized, placebo-controlled Phase 2b clinical study in patients with SLE (the CARE study; FIG. 1A). In IFN-1 high populations reports have shown an association with renal aspects of disease, low complement C3 and autoantibodies, in keeping with the molecular function of IFNs [Landolt-Marticorena, C. et al., Ann Rheum Dis 2009, 68, 1440-1446, PMID:18772188; Feng, X. et al., Arthritis Rheumatism 2006, 54(9), 2951-2962, PMID:16947629; Kirou, K A. et al., Arthritis Rheumatism, 2005, 52(5), 1491-1503, PMID:15880830]. In a longitudinal study, patients with a high type 1 interferon (IFN-1) gene signature score (“IFN-1 high patients”) were more likely to be receiving higher immunosuppressants and glucocorticoids than IFN-1 low patients (75% vs 50%), were likely to be younger and of Asian ancestry [Northcott, M. et al., Lupus Sci Med 2022, 9, e000625, PMID:35197305]. The effect of Glucocorticoids on IFN gene expressions was evaluated in a small cohort of SLE patients. The authors saw no effect of glucocorticoid dose on type 1 interferon-regulated gene expression. Patients with a high type 1 interferon gene signature had reduced glucocorticoid gene signature expression compared with patients with a low type 1 interferon gene signature matched for glucocorticoid dose, suggesting type 1 interferon inhibits glucocorticoid-stimulated gene expression [Northcott, M. et al., Lancet Rheumatology 2021, 3(5), e357-e370, DOI: 10.1016/S2665-9913(21)00006-0].

The prominent role of IFNs in SLE has prompted efforts to target this pathway as a means of treating SLE and has been investigated in clinical trials. A prime example is anifrolumab, an antibody targeting the IFN-1 receptor. In addition, further potential therapies have been investigated for their potential ability to modulate IFN-1 gene signature score.

The phase 3 studies of anifrolumab confirmed the suppression of the interferon signature in patients with IFN-1 high at baseline seen in a phase 2 trial, although the association of this effect with clinical efficacy was not demonstrated. In TULIP 1 the efficacy of anifrolumab measured with a (SLE) Responder Index (SRI-4) [Furie, R. et al. Arthritis Rheumatism 2009, 61(9), 1143-1151, PMID: 19714615] (placebo adjusted) in patients with IFN-1 high at baseline is modest, i.e., 6 percentage points. In TULIP 2 the efficacy of anifrolumab (placebo adjusted) measured with SRI-4 is numerically higher in patients with IFN-1 high at baseline than in patients with IFN-1 low, i.e., 17.3 and 11.2 percentage points, respectively. The TULIP 2 manuscript authors indicated that because the number of IFN-1 low patients was small (30 in low vs 150 in high, 17%), the effect of anifrolumab in such patients requires further analysis. The pooled phase 3 studies TULIP 1 & 2 allows comparisons of greater sample size for the IFN-1 low group, (i.e., 122 patients in IFN-1 low treated with 300 mg or placebo, 60 and 62 patients, respectively) [Vital, E M. et al., Ann Rheum Dis 2022, 81, 951-961, PMID:35338035]. There was no treatment effect on SRI-4 in the IFN-1 low group (45.2% and 45.3% for the 300 mg anifrolumab and placebo, respectively) while a modest effect of 9 percentage points on BICLA (British Isles Lupus Assessment Group-based Composite Lupus Assessment) was observed (46.8% and 37.5%, for the 300 mg anifrolumab and placebo, respectively.

The FDA analysis of the NDA on anifrolumab 150, 300 and 1000 mg led to their conclusion “that while neutralization of the IFN-1 gene signature reflects the direct pharmacological effect of anifrolumab and there is a clear dose-dependent PD response, its clinical relevance is still unclear as the PD response is higher in the non-responders on the 300 mg arm compared to the responders on the 150 mg arm” [FDA SAPHNELO multi-discipline review, p 44-47 (CENTER FOR DRUG EVALUATION AND RESEARCH, BLA 761123 Multi-disciplinary Review and Evaluation, Saphnelo (anifrolumab-fnia) for adults with SLE, version date: Oct. 12, 2018)]. This is based on the FDA analysis of the PD response (neutralization of Type-1 gene signature and responder/non-responder analysis for SRI-4 and BICLA).

In summary, while AZ indicates that further studies are warranted to investigate the difference in efficacy between IFN-1 high and low population, the FDA gave a broad SLE population label indication. This suggests that it is not obvious that by targeting the IFN type 1 pathway specifically, you have a benefit only in IFN-high patients but also have a benefit in IFN-low patients too. Conversely, it is not obvious that by targeting mechanisms not directly related to modulating the type 1 interferon pathway one could expect to see only a beneficial effect in IFN-high patients with SLE but not in IFN-low patients which is what is observed with cenerimod (FIG. 1c).

Iberdomide is a high affinity cereblon ligand that promotes proteasomal degradation of transcription factors Ikaros and Aiolos. Aiolos is a B cell modulator and is required for maturation of plasma cells. Ikaros is required for development of B cells and plasmacytoid pDCs, which are important producers of IFN-α [Lipsky, P E. et al., Ann Rheum Dis 2022, 81, 1136-1142, PMID:35477518].

Iberdomide doses of 0.15, 0.30 and 0.45 mg were investigated against placebo in a phase 2 study where 179 of 288 patients (62%) were IFN-1 high at baseline. This trial was not stratified according to gene signature at baseline, the highest proportion of IFN-1 high was in the 0.45 mg group (70%), and the lowest in the placebo group (58%) [Merrill, J T. et al., N Engl J Med 2022, 386, 1034-1045, PMID:35294813].

At week 24, the highest SRI-4 response (the primary end point) had occurred in 44 of 81 patients (54%) receiving the 0.45-mg dose of iberdomide and in 29 of 83 patients (35%) receiving placebo (adjusted difference, 19.4 percentage points). Iberdomide and placebo did not have meaningful differences with respect to most secondary end points. Among the patients with an IFN-1 high status, the responses occurred in 34 of 57 patients (60%) in the 0.45 mg iberdomide group vs 16 of 48 patients (33%) in the placebo group. Even if exploratory analysis indicates that the highest cut point for the IFN-1 subgroup (representing 31% of the total study population) was associated with the most enhanced relationship with response, providing a treatment difference of 54 percentage points vs placebo (5 out of 27 (20.0%) vs 17 out of 23 (74%)) [Lipsky, P E. et al., Ann Rheum Dis 2022, 81, 1136-1142, PMID:35477518], still 10 of 24 patients (42%) with an IFN-1 low status responded to iberdomide 0.45 mg. Analysis of changes in the biomarkers showed that the phase 2 SLE patients with an IFN-1 high status at baseline manifested a significant reduction in the IFN signature as a result of active treatment, whereas those with low baseline IFN signature did not [[Lipsky, P E. et al., Ann Rheum Dis 2022, 81, 1136-1142, PMID:35477518]. Treatment with iberdomide was associated with a significant, dose-dependent reduction in pDCs and mDCs, which are thought to be primary sources of type 1 IFNs.

Rontalizumab, despite targeting the IFN-1 pathway, was found to be more efficacious in IFN-1 low patients (Kalunian, K. et al., Ann Rheum Dis 2016, 75, 196-202, PMID: 26038091). This further highlights the complexity to identify responder patients' subgroups.

Sifalimumab is a fully human, immunoglobulin G1 κ monoclonal antibody that binds to and neutralizes most IFN-α subtypes. It was developed by AstraZeneca and its development was discontinued in favor of anifrolumab. Earlier clinical trials of sifalimumab have established its suppression of IFN-α-induced genes and have suggested favorable effects on clinical outcome measures [Petri, M. et al., Arthritis Rheumatism 2013, 65(4), 1011-1021, PMID:23400715].

Despite extensive investigations into the IFN pathway and its association with SLE, to date the association of S1P1 receptor modulation and IFN signalling remains poorly explored. Early evidence linking IFN alpha with S1P1 was published in 2006 (Shiow, L. et al., Nature 2006, 440, 540-544, PMID: 16525420). Here it was reported that mice treated with poly(I:C), a double-stranded RNA mimetic, which induces IFN signalling, lead to CD69 modulation and the subsequent inhibition of lymphocyte egress from lymph nodes via a down-regulation of the S1P1 signalling pathway. This suggested that IFN signalling could modulate S1P1 mediated lymphocyte migration. Teijaro (Teijaro, J R. et al., PNAS 2016, 113(5), 1351-1356, PMID: 26787880) further reported that sphingosine 1-phosphate (S1P)-S1PR1 signaling in plasmacytoid dendritic cells (pDCs) directly inhibits IFN-α auto-amplification by induced degradation of the interferon alpha receptor 1 (IFNAR1) receptor, again suggesting that an S1P1 receptor modulator could lead to a decrease in IFN mediated signalling. However, in mouse models of influenza it was reported that S1P1 receptor signaling on pulmonary endothelial cells in vivo blunts but does not abolish IFN-a production [Teijaro, J R. et al., Cell 2011, 146(6), 980-991, PMID:21925319].

The potential role of S1P modulators in modulating IFN associated proteins in the context of SLE was recently investigated by Strasser et al. Efficacy in an MRL/lpr mouse model of SLE was demonstrated with a decrease in lymphocytes in the periphery and an improvement in survival. Further, a small exploratory study involving 13 patients diagnosed with SLE were treated with cenerimod for a period of 12 weeks. A large heterogeneity in IFN associated biomarkers between the patients in the study was observed and cenerimod treatment led to a trend in the reduction of IFN associated biomarkers [Strasser, D S et al., RMD Open 2020, 6, e001261, PMID: 32917831].

Together these data suggest a complex and contradictory interaction between the IFN-1 and S1P1 signalling pathways in the context of disease. There was no previous evidence demonstrating that S1P1 modulation can lead to an improvement in the health of patients with SLE, as measured by mSLEDAI-2K, who exhibit specifically an IFN-1 high gene signature and elevated IFN associated proteins.

### DESCRIPTION OF THE FIGURES

FIG. 1 shows:
A) A histogram showing the distribution of the type 1 IFN gene signature score at baseline from the CARE study of the combined placebo and cenerimod 4 mg cohorts. Clearly visible is a bimodal distribution. The grey line depicts the cut-off between IFN-1 low and IFN-1 high at −0.5.
B) Boxplot depicting individual (IFN-1 high) patients represented as dots at baseline and at month 6 of treatment for the placebo and the 4 mg cenerimod treated groups from the CARE study showing the type 1 IFN gene signature score (IFN-1 score) that was measured for each individual from blood. In boxplots, the whiskers indicate the minimum and maximum range. A decrease in the mean value of the IFN-1 score in the cenerimod 4 mg group is observed after 6 months compared to baseline, whereas there is a very small difference observed in the mean value is observed over 6 months in the placebo group.
C) Graph showing the least square (LS) mean change from baseline to 6 months in modified SLEDAI-2K (mSLEDAI-2K). The upper graph shows patients who were classified as IFN-1 high at baseline and compares placebo with cenerimod 4 mg up to 6 months. The lower panel shows patients who were classified as IFN-1 low at baseline and compares placebo with cenerimod 4 mg. No difference is observed between placebo and cenerimod 4 mg in the IFN-1 low groups in mSLEDAI-2K at 6 months. A clear difference is observed in the IFN-1 high group in the decrease observed between cenerimod 4 mg and placebo at 6 months compared to baseline.

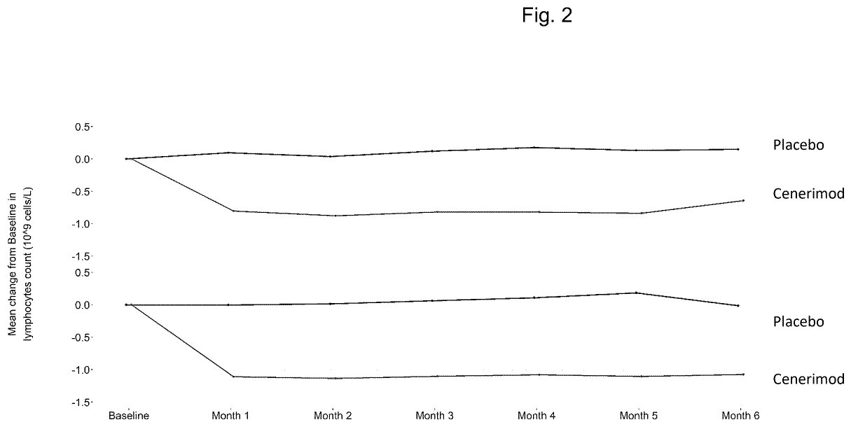

FIG. 2 shows:
Effect of placebo and cenerimod 4 mg on blood lymphocyte counts over the treatment period of 6 months. The mean change from baseline in lymphocyte count is displayed. Whereas placebo does not decrease blood lymphocyte counts, a fast and sustained decrease is observed both in the IFN-1 high and IFN-1 low patient subgroups treated with cenerimod 4 mg.

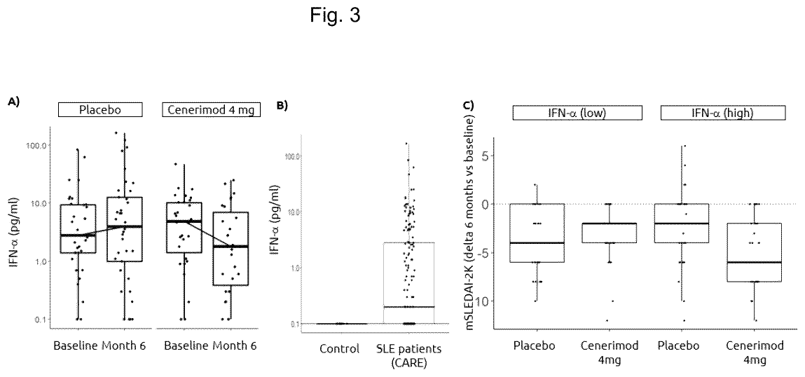

FIG. 3 shows:
A) boxplot depicting individual patients represented as dots at baseline and at month 6 of treatment for the placebo and the 4 mg cenerimod treated groups from the CARE study showing the amount of IFN alpha protein in pg/ml that was measured for each individual from blood plasma. In boxplots, the whiskers indicate the minimum and maximum range. A decrease in the mean value of IFN alpha protein in the cenerimod 4 mg group is observed after 6 months compared to baseline, whereas a slight elevation in the mean value is observed over 6 months in the placebo group.
B) IFN alpha protein in plasma in pg/ml from a healthy volunteer cohort (control) and from the placebo and 4 mg cenerimod cohorts from the CARE study at baseline depicted in a boxplot. Individual patients are represented as dots. 95% of healthy volunteers have values below 0.1 pg/ml and this is represented by the line shown. This marks the cutoff point between high and low. The patient cohort has a higher level of IFN alpha protein than the healthy volunteer cohort.
C) The delta of the mSLEDAI-2K values at 6 months compared to baseline are depicted for the placebo and cenerimod 4 mg cohorts from the CARE study as boxplots. The placebo and cenerimod 4 mg cohorts are segregated into IFN alpha low and IFN alpha high groups according to their measured IFN alpha values at baseline. The decrease in mSLEDAI-2K score (i. e an improvement in the clinical readout) is similar for both the placebo and cenerimod 4 mg IFN alpha protein low groups. The decrease in mSLEDAI-2K for the placebo IFN high group is small. However, the cenerimod 4 mg IFN alpha protein high group shows a marked reduction in mSLEDAI-2K when compared to the other 3 groups.

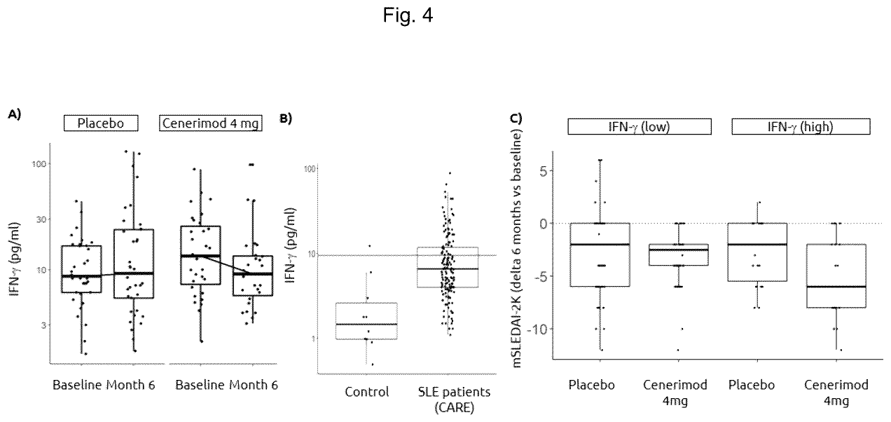

FIG. 4 shows:
A) boxplot depicting individual patients represented as dots at baseline and at month 6 of treatment for the placebo and the 4 mg cenerimod treated groups from the CARE study showing the amount of the IFN gamma protein in pg/ml that was measured for each individual from blood plasma. In boxplots, the whiskers indicate the minimum and maximum range. A decrease in the mean value of IFN gamma protein in the cenerimod 4 mg group is observed between baseline and after 6 months treatment, whereas a slight elevation in the mean value is observed over 6 months in the placebo group.
B) IFN gamma protein in plasma in pg/ml from a healthy volunteer cohort (control) and from the placebo and 4 mg cenerimod cohorts from the CARE study at baseline depicted in a boxplot. Individual patients are represented as dots. 95% of healthy volunteers have values below 9.5 pg/ml and this is represented by the line shown. This marks the cut-off point between high and low. The patient cohort has a higher level of IFN gamma protein than the healthy volunteer cohort.
C) The delta of the mSLEDAI-2K values at 6 months compared to baseline are depicted for the placebo and cenerimod 4 mg cohorts from the CARE study as boxplots. The placebo and cenerimod 4 mg cohorts are segregated into IFN gamma low and IFN gamma high groups according to their measured IFN gamma values at baseline. The decrease in mSLEDAI-2K score (i.e., an improvement in the clinical readout) is similar for both the placebo and cenerimod 4 mg IFN gamma protein low groups. The decrease in mSLEDAI-2K for the placebo IFN gamma high group is small. However, the cenerimod 4 mg IFN gamma high group shows a marked reduction in mSLEDAI-2K when compared to the other 3 groups.

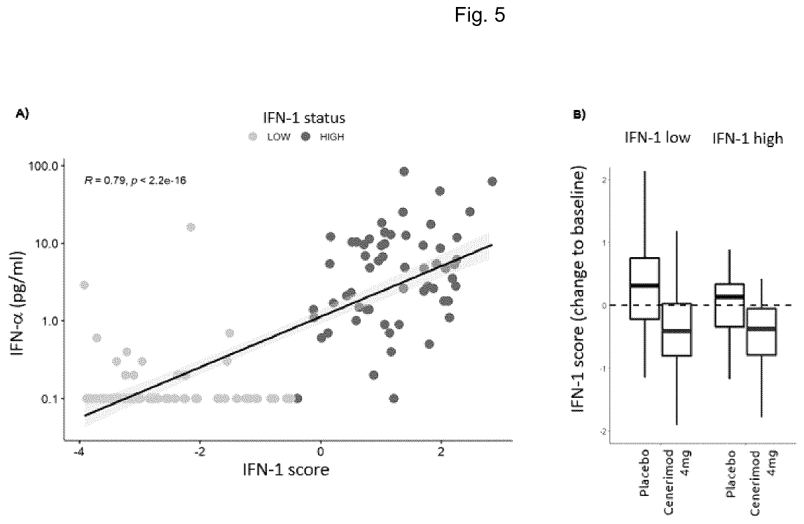

FIG. 5 shows:
A significant correlation of plasma IFN-α levels with the blood IFN-1 gene expression score was observed (A). Cenerimod 4 mg treatment for 6 months decreased the IFN-1 gene expression score both in IFN-1 high and low status patients. In contrast, placebo treatment for 6 months rather increased the IFN-1 gene expression score both in IFN-1 high and low status patients (B).

### DETAILED DESCRIPTION OF THE INVENTION

Various embodiments of the invention are presented hereafter:
1) A first embodiment of the invention relates to a S1P₁ receptor modulator, or a pharmaceutically acceptable salt thereof, for use in the prevention or treatment (especially treatment) of a type 1 interferon mediated disease in a subject, wherein the subject has a high type 1 interferon (IFN-1) gene signature score.

The term “has” in the context of “the subject has a high type 1 interferon (IFN-1) gene signature score”, refers to a subject that has and/or that is identified as having and/or that diagnosed as having a high type 1 interferon (IFN-1) gene signature score.
2) A further embodiment of the invention relates to a S1P₁ receptor modulator, or a pharmaceutically acceptable salt thereof, for use according to embodiment 1), wherein the subject has a high type 1 interferon (IFN-1) gene signature score at treatment onset.
3) A further embodiment of the invention relates to a S1P₁ receptor modulator, or a pharmaceutically acceptable salt thereof, for use according to any one of embodiments 1) or 2), wherein the type 1 interferon gene signature score is calculated based on any one of the following groups of type 1 interferon-associated genes:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) IFI6, RSAD2, IFI44, IFI44L, and IFI27; or
d) IFI6, RSAD2, IFI44, and IFI44L; or
e) IFI6, RSAD2, IFI44L, and IFI27; or
f) IF16, RSAD2, IFI44, and IFI27; or
g) RSAD2, IFI44, IFI44L, and IFI27; or
h) IFI6, IFI44, IFI44L, and IFI27; or
i) IFI6, MX1, IFIT1, and HERC5; or
j) EIF2AK2, MX1, and IFIT1; or
k) EIF2AK2, IFI44, and IFIT1.
l) EIF2AK2, IFI6, RSAD2, IFI44, IFI44L, IFI27, MX1, IFIT1, HERC5, and ISG15; or
m) RSAD2, IFI27, IFIT1, and HERC5.

Group a) is disclosed in WO 2020/084591; group b) in WO 2022/074123 and in Yao Y, et al. Human Genomics and Proteomics 2009; article ID 374312, doi:10.4061/2009/374312; groups c) to h) in WO 2011/028933 and in Furie R, et al. Arthritis & Rheumatology 2017; 69(2) 376-386; group i) in Westra H-J, et al. Nature Genetics 2013; 45(10) 1238-1243, doi:10.1038/ng.2756; group j) in Niewold, T et al. Genes and Immunity 2007; 8 492-502, doi:10.1038/sj.gene.6364408; group k) in Kirou, K. et al. Arthritis & Rheum. 2005; 52(5) 1491-1503; and groups l) and m) in Northcott M, et al. Lupus Science & Medicine 2022; 9: e000625; doi:10.1136/lupus-2021-000625.
4) A further embodiment of the invention relates to a S1P₁ receptor modulator, or a pharmaceutically acceptable salt thereof, for use according to any one of embodiments 1) or 2), wherein the type 1 interferon gene signature score is calculated based on any one of the following groups of type 1 interferon-associated genes:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) RSAD2, IFI44, IFI44L, and IFI27; or
d) EIF2AK2, IFI6, RSAD2, IFI44, IFI44L, IFI27, MX1, IFIT1, HERC5, and ISG15; or
e) RSAD2, IFI27, IFIT1, and HERC5.
5) A further embodiment of the invention relates to a S1P₁ receptor modulator, or a pharmaceutically acceptable salt thereof, for use according to any one of embodiments 1) or 2), wherein the type 1 interferon gene signature score is calculated based on any one of the following groups of type 1 interferon-associated genes:
a) RSAD2, IFI44, IFI44L, and IFI27; or
b) EIF2AK2, IFI6, RSAD2, IFI44, IFI44L, IFI27, MX1, IFIT1, HERC5, and ISG15; or
c) RSAD2, IFI27, IFIT1, and HERC5.
6) A further embodiment of the invention relates to a S1P₁ receptor modulator, or a pharmaceutically acceptable salt thereof, for use according to any one of embodiments 1) or 2), wherein the type 1 interferon gene signature score is calculated based on any one of the following groups of type 1 interferon-associated genes:
a) RSAD2, IFI44, IFI44L, and IFI27; or
b) RSAD2, IFI27, IFIT1, and HERC5.
7) A further embodiment of the invention relates to a S1P₁ receptor modulator, or a pharmaceutically acceptable salt thereof, for use according to any one of embodiments 1) or 2), wherein the type 1 interferon gene signature score is calculated based on the type 1 interferon-associated genes EIF2AK2, IFI6, RSAD2, IFI44, IFI44L, IFI27, MX1, IFIT1, HERC5, and ISG15.
8) A further embodiment of the invention relates to a S1P₁ receptor modulator, or a pharmaceutically acceptable salt thereof, for use according to any one of embodiments 1) or 2), wherein the type 1 interferon gene signature score is calculated based on the type 1 interferon-associated genes RSAD2, IFI27, IFIT1, and HERC5.

The type 1 interferon gene signature score of a subject can be calculated according to any of the methods given in WO 2011/028933, WO 2020/084591, Furie R, et al. Arthritis & Rheumatology 2017; 69(2) 376-386 or Northcott M, et al. Lupus Science & Medicine 2022; 9:e000625; doi:10.1136/lupus-2021-000625. Northcott et al. describes especially a method to calculate the IFN-1 gene signature score of a subject based on the 4 genes RSAD2, IFI27, IFIT1, and HERC5 and further discloses an IFN-1 gene signature score cut-off value of −0.5 between subjects having a low and a high IFN-1 gene signature score.

Northcott et al. further refers to an IFN-1 test that is commercially available from DxTerity Diagnostics Inc. Especially, the type 1 interferon gene signature score of a subject can be calculated by the method originally developed by DxTerity Diagnostics Inc. by (i) measuring the gene (RNA) expression levels of the type 1 interferon-associated genes RSAD2, IFI27, IFIT1, and HERC5 in a blood sample of a subject (for instance by measuring respective peak height in capillary electrophoresis electropherograms); (ii) measuring the gene expression levels of the three housekeeping (normalizer) genes ACTB, GAPDH, and TFRC in a blood sample of the subject; (iii) calculating the binary logarithm (log₂) of the expression levels of each of the three housekeeping genes and the arithmetic mean thereof; (iv) calculating the normalized expression for each of the four type 1 interferon-associated genes according to Formula (I); and (v) calculating the type 1 interferon gene signature score by averaging the normalized expression values of the four type 1interferon-associated genes.

Normalized Expression Gene i=Log₂ (Height Gene i)−Mean (Log₂ (Normalizer Genes Height)) Formula (I)

An important property of the IFN-1 gene signature is the bimodal distribution in subjects with systemic lupus erythematosus (SLE) allowing separation into subjects with a high type 1 interferon (IFN-1) gene signature score (IFN-1 high subjects) and subjects with a low score (IFN-1 low subjects) [El-Sherbiny, Y M. et al., Scientific Reports 2018, 8, 5793, DOI:10.1038/s41598-018-24198-1, PMID: 29643425]. Subjects having a high IFN-1 gene signature score can be defined as subjects with an IFN-1 gene signature score above a threshold value (or alternatively “cut-off value”); those having a low IFN-1 gene signature score can be defined as subjects with an IFN-1 gene signature score at or below the threshold value. The threshold or cut-off value can be defined in accordance with any of the methods known in the art (for instance WO 2011/028933 or Northcott M, et al. Lupus Science & Medicine 2022; 9:e000625; doi:10.1136/lupus-2021-000625). Especially the threshold/cut-off value can be defined relative to the IFN-1 gene signature score of healthy humans: for instance, the threshold/cut-off value may be any IFN-1 gene signature score value between 1.8 to 4.0 (notably 2.0 to 3.0) standard deviations above the arithmetic mean value from at least 20 (notably at least 200) healthy humans. Notably, the threshold/cut-off value of the IFN-1 gene signature score may be defined as 2 standard deviations above the arithmetic mean value from at least 100 (notably at least 300) healthy humans. Notably, the IFN-1 gene signature scores of subjects (especially patients) and of healthy humans (for determination of the cut-off value) are calculated using the same method of IFN-1 gene signature score determination.

Abedi et al. (DxTerity) have compared in a study the relative performance of four different IFN gene signatures in a cohort of 687 participants with self-reported systemic lupus erythematosus (SLE). The frequently used 4-gene IFN signature of IFI27, IFI44, IFI44L, and RSAD2 identified 36.5% of the participants as IFN high and 63.5% as IFN low. Three other literature reported IFN signatures (IFI6, MX1, IFIT1, and HERC5; EIF2AK2, MX1, and IFIT1; and EIF2AK2, IFI44, and IFIT1) provided similar classification results with participants being assigned to the same IFN sub-group (IFN high/IFN low) over 90% of the time, and nearly identical patient distributions; all four IFN gene signatures resulted in a similar bimodal IFN-1 gene signature score distribution as found with the 4 genes RSAD2, IFI27, IFIT1, and HERC5 and shown in FIG. 1A (Abedi M et al. Annals of the Rheumatic Diseases 2018; 77 (Suppl. 2: EULAR 2018 abstracts) 885-886 (#SAT0041), DOI: 10.1136/annrheumdis-2018-eular.3758 and corresponding poster: Abedi M, Comparison Of Different Type 1 IFN Signatures Demonstrates Concordance In A Real World, Home Monitored Systemic Lupus Erythematosus Cohort, EULAR 2018, poster #SAT0041).
9) A further embodiment of the invention relates to a S1P₁ receptor modulator, or a pharmaceutically acceptable salt thereof, for use according to any one of embodiments 1) to 8), wherein the type 1 interferon gene signature score of the subject is at or above the minimum of the bimodal distribution curve of the type 1 interferon gene signature scores of SLE patients.

The minimum of the bimodal distribution curve may be obtained by determining the number of SLE patients with a given type 1 interferon gene signature score as a function of the type 1 interferon gene signature score. The bimodal distribution curve may be based on the type 1 interferon gene signature scores of at least 50, of at least 100, of at least 200, or (preferably) of at least 300 SLE patients. The bimodal distribution curve may be obtained from current (e.g., from data collected less than 1 year ago) and/or from historical (e.g., from data collected more than 1 year ago) data on type 1 interferon gene signature scores in SLE patients.
10) A further embodiment of the invention relates to a S1P₁ receptor modulator, or a pharmaceutically acceptable salt thereof, for use according to embodiment 9), wherein the type 1 interferon gene signature score of the subject is at least 0.5 units, at least 1 unit, at least 2 units or at least 3 units (especially at least 1 unit) above the minimum of the bimodal distribution curve of the type 1 interferon gene signature scores of SLE patients.
11) A further embodiment of the invention relates to a S1P₁ receptor modulator, or a pharmaceutically acceptable salt thereof, for use according to any one of embodiments 1) to 8), wherein the subject has a type 1 interferon gene signature score that is higher than the type 1 interferon gene signature score of a (representative) healthy subject.
12) A further embodiment of the invention relates to a S1P₁ receptor modulator, or a pharmaceutically acceptable salt thereof, for use according to any one of embodiments 1) to 8), wherein the type 1 interferon gene signature score of the subject is above a threshold value.
13) A further embodiment of the invention relates to a S1P₁ receptor modulator, or a pharmaceutically acceptable salt thereof, for use according to embodiment 12), wherein the threshold value is an IFN-1 gene signature score value that is selected from values between 1.8 to 4.0 (i.e. 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0; especially 2.0 to 3.0, and notably 2.0 to 2.5) standard deviations above the arithmetic mean of the IFN-1 gene signature score values from at least 20, at least 50, at least 100, at least 200, at least 300 or (preferably) at least 500 healthy humans.
14) A further embodiment of the invention relates to a S1P₁ receptor modulator, or a pharmaceutically acceptable salt thereof, for use according to embodiment 12), wherein the threshold value of the IFN-1 gene signature score is 2 standard deviations above the arithmetic mean value from at least 100, at least 200, at least 300 or (preferably) at least 500 healthy humans.
15) A further embodiment of the invention relates to a S1P₁ receptor modulator, or a pharmaceutically acceptable salt thereof, for use according to embodiment 12), wherein the threshold value of the IFN-1 gene signature score is between- 1.0 and 0 (especially about −0.5).
16) A further embodiment of the invention relates to a S1P₁ receptor modulator, or a pharmaceutically acceptable salt thereof, for use according to embodiment 12), wherein the threshold value of the IFN-1 gene signature score is −0.5.
17) A further embodiment of the invention relates to a S1P₁ receptor modulator, or a pharmaceutically acceptable salt thereof, for use according to any one of embodiments 1) to 16), wherein the type 1 interferon gene signature score of the subject is calculated (determined) by a method comprising the following steps:
(i) measuring the gene (RNA) expression levels of the type 1 interferon-associated genes (especially of RSAD2, IFI44, IFI44L, and IFI27; or of RSAD2, IFI27, IFIT1, and HERC5; and notably of RSAD2, IFI27, IFIT1, and HERC5) in a blood sample of a subject (notably by measuring respective peak height by capillary electrophoresis);
(ii) measuring the gene expression levels of the three housekeeping (normalizer) genes ACTB, GAPDH, and TFRC in a blood sample of the subject;
(iii) calculating the binary logarithm (log₂) of the expression levels of each of the three housekeeping genes and the arithmetic mean thereof;
(iv) calculating the normalized expression for each of the type 1 interferon-associated genes according to Formula (I):

Normalized Expression Gene i=Log₂ (Height Gene i)−Mean (Log₂ (Normalizer Genes Height)) Formula (I)
and (v) calculating the type 1 interferon gene signature score by averaging the normalized expression values of the type 1 interferon-associated genes.

18) A further embodiment of the invention relates to a S1P₁ receptor modulator, or a pharmaceutically acceptable salt thereof, for use according to any one of embodiments 3) to 16), wherein the type 1 interferon gene signature score of the subject is calculated (determined) by a method comprising the following steps:
(i) measuring the gene (RNA) expression levels of the type 1 interferon-associated genes (especially of RSAD2, IFI44, IFI44L, and IFI27; or of RSAD2, IFI27, IFIT1, and HERC5; and notably of RSAD2, IFI27, IFIT1, and HERC5) in a blood sample of a subject (notably by measuring respective peak height by capillary electrophoresis);
(ii) measuring the gene expression levels of the three housekeeping (normalizer) genes ACTB, GAPDH, and TFRC in a blood sample of the subject;
(iii) calculating the binary logarithm (log₂) of the expression levels of each of the three housekeeping genes and the arithmetic mean thereof;
(iv) calculating the normalized expression for each of the four type 1 interferon-associated genes according to Formula (I):

Normalized Expression Gene i=Log₂ (Height Gene i)−Mean (Log₂ (Normalizer Genes Height)) Formula (I)
and (v) calculating the type 1 interferon gene signature score by averaging the normalized expression values of the four type 1 interferon-associated genes.
19) A further embodiment of the invention relates to a S1P₁ receptor modulator, or a pharmaceutically acceptable salt thereof, for use in a method of preventing or treating (especially treating) a subject having a type 1 interferon mediated disease (especially a type 1 interferon mediated disease that is responsive to treatment with the S1P₁ receptor modulator), the method comprising:
a) providing a biological sample (especially a blood sample or a serum sample, and notably a blood sample) from the subject;
b) assaying gene expression of at least three (especially at least four) type 1 interferon-associated genes selected from the group consisting of DHX58, EIF2AK2, IRF7, PARP9, SAMD9L, IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6 (especially IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6, and notably EIF2AK2, IFI6, RSAD2, IFI44, IFI44L, IFI27, MX1, IFIT1, HERC5, and ISG15) in the biological sample;
c) determining a normalised expression value of each assayed gene;
d) calculating a type 1 interferon gene signature score by averaging the normalised expression values; and
e) treating the subject with the S1P₁ receptor modulator when the type 1 interferon gene signature score of the subject is higher than a threshold value.
20) A further embodiment of the invention relates to a S1P₁ receptor modulator, or a pharmaceutically acceptable salt thereof, for use according to embodiment 19), wherein the subject has a type 1 interferon gene signature score that is higher than a threshold value at treatment onset and/or is higher than the type 1 interferon gene signature score in a biological sample from a healthy subject at treatment onset.

Especially, the subject has a type 1 interferon gene signature score that is higher than a threshold value at treatment onset.
21) A further embodiment of the invention relates to a S1P₁ receptor modulator, or a pharmaceutically acceptable salt thereof, for use according to any one of embodiments 19) or 20), wherein the biological sample is a blood sample.
22) A further embodiment of the invention relates to a S1P₁ receptor modulator, or a pharmaceutically acceptable salt thereof, for use according to any one of embodiments 19) to 21), wherein one of the following groups of type 1 interferon-associated genes is selected for assaying gene expression:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) IFI6, RSAD2, IFI44, IFI44L, and IFI27; or
d) IFI6, RSAD2, IFI44, and IFI44L; or
e) IFI6, RSAD2, IFI44L, and IFI27; or
f) IFI6, RSAD2, IFI44, and IFI27; or
g) RSAD2, IFI44, IFI44L, and IFI27; or
h) IFI6, IFI44, IFI44L, and IFI27; or
i) IFI6, MX1, IFIT1, and HERC5; or
j) EIF2AK2, MX1, and IFIT1; or
k) EIF2AK2, IFI44, and IFIT1.
l) EIF2AK2, IFI6, RSAD2, IFI44, IFI44L, IFI27, MX1, IFIT1, HERC5, and ISG15; or
m) RSAD2, IFI27, IFIT1, and HERC5.

23) A further embodiment of the invention relates to a S1P₁ receptor modulator, or a pharmaceutically acceptable salt thereof, for use according to any one of embodiments 19) to 21), wherein one of the following groups of type 1 interferon-associated genes is selected for assaying gene expression:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) RSAD2, IFI44, IFI44L, and IFI27; or
d) EIF2AK2, IFI6, RSAD2, IFI44, IFI44L, IFI27, MX1, IFIT1, HERC5, and ISG15; or
e) RSAD2, IFI27, IFIT1, and HERC5.
24) A further embodiment of the invention relates to a S1P₁ receptor modulator, or a pharmaceutically acceptable salt thereof, for use according to any one of embodiments 19) to 21), wherein one of the following groups of type 1 interferon-associated genes is selected for assaying gene expression:
a) RSAD2, IFI44, IFI44L, and IFI27; or
b) EIF2AK2, IFI6, RSAD2, IFI44, IFI44L, IFI27, MX1, IFIT1, HERC5, and ISG15; or
c) RSAD2, IFI27, IFIT1, and HERC5.
25) A further embodiment of the invention relates to a S1P₁ receptor modulator, or a pharmaceutically acceptable salt thereof, for use according to any one of embodiments 19) to 21), wherein one of the following groups of type 1 interferon-associated genes is selected for assaying gene expression:
a) RSAD2, IFI44, IFI44L, and IFI27; or
b) RSAD2, IFI27, IFIT1, and HERC5.
26) A further embodiment of the invention relates to a S1P₁ receptor modulator, or a pharmaceutically acceptable salt thereof, for use according to any one of embodiments 19) to 21), wherein the type 1 interferon-associated genes for assaying gene expression are EIF2AK2, IFI6, RSAD2, IFI44, IFI44L, IFI27, MX1, IFIT1, HERC5, and ISG15.
27) A further embodiment of the invention relates to a S1P₁ receptor modulator, or a pharmaceutically acceptable salt thereof, for use according to any one of embodiments 19) to 21), wherein the type 1 interferon-associated genes for assaying gene expression are RSAD2, IFI27, IFIT1, and HERC5.
28) A further embodiment of the invention relates to a S1P₁ receptor modulator, or a pharmaceutically acceptable salt thereof, for use according to any one of embodiments 19) to 27), wherein the gene expression is assayed by capillary electrophoresis (especially capillary electrophoresis suitable for nucleotide analysis).
29) A further embodiment of the invention relates to a S1P₁ receptor modulator, or a pharmaceutically acceptable salt thereof, for use according to any one of embodiments 19) to 27), wherein the gene expression of the type 1 interferon-associated genes is assayed by measuring peak height in capillary electrophoresis electropherograms.
30) A further embodiment of the invention relates to a S1P₁ receptor modulator, or a pharmaceutically acceptable salt thereof, for use according to any one of embodiments 19) to 29), wherein the normalized expression values are determined by (i) measuring the gene expression levels of the three housekeeping (normalizer) genes ACTB, GAPDH, and TFRC in a blood sample of the subject; (ii) calculating the binary logarithm (log₂) of the expression levels of each of the three housekeeping genes and the arithmetic mean thereof; and (iii) calculating the normalized expression value for each of the type 1 interferon-associated genes according to Formula (II)

Normalized Expression Gene i=Log₂ (Expression Level of Gene i)−Mean (Log₂ (Expression Level of Normalizer Genes)) Formula (II).

In a preferred embodiment the normalized expression values are calculated based on peak heights (especially peak heights in capillary electrophoresis electropherograms) of the respective genes by using Formula (I).
31) A further embodiment of the invention relates to a S1P₁ receptor modulator, or a pharmaceutically acceptable salt thereof, for use according to any one of embodiments 19) to 30), wherein the type 1 interferon gene signature score of the subject is at or above the minimum of the bimodal distribution curve of the type 1 interferon gene signature scores of SLE patients.

The minimum of the bimodal distribution curve may be obtained by determining the number of SLE patients with a given type 1 interferon gene signature score as a function of the type 1 interferon gene signature score. The bimodal distribution curve may be based on the type 1 interferon gene signature scores of at least 50, of at least 100, of at least 200, or (preferably) of at least 300 SLE patients. The bimodal distribution curve may be obtained from current (e.g., from data collected less than 1 year ago) and/or from historical (e.g., from data collected more than 1 year ago) data on type 1 interferon gene signature scores in SLE patients.
32) A further embodiment of the invention relates to a S1P₁ receptor modulator, or a pharmaceutically acceptable salt thereof, for use according to embodiment 31), wherein the type 1 interferon gene signature score of the subject is at least 0.5 units, at least 1 unit, at least 2 units or at least 3 units (especially at least 1 unit) above the minimum of the bimodal distribution curve of the type 1 interferon gene signature scores of SLE patients.
33) A further embodiment of the invention relates to a S1P₁ receptor modulator, or a pharmaceutically acceptable salt thereof, for use according to any one of embodiments 19) to 30), wherein the threshold value is an IFN-1 gene signature score value that is selected from values between 1.8 to 4.0 (i.e. 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0; especially 2.0 to 3.0, and notably 2.0 to 2.5) standard deviations above the arithmetic mean of the IFN-1 gene signature score values from at least 20, at least 50, at least 100, at least 200, at least 300 or (preferably) at least 500 healthy humans.
34) A further embodiment of the invention relates to a S1P₁ receptor modulator, or a pharmaceutically acceptable salt thereof, for use according to any one of embodiments 19) to 30), wherein the threshold value of the IFN-1 gene signature score is 2 standard deviations above the arithmetic mean value from at least 100, at least 200, at least 300 or (preferably) at least 500 healthy humans.
35) A further embodiment of the invention relates to a S1P₁ receptor modulator, or a pharmaceutically acceptable salt thereof, for use according to any one of embodiments 19) to 30), wherein the threshold value of the IFN-1 gene signature score is selected from values between −1.0 and 0 (especially about −0.5, and notably −0.5).
36) A further embodiment of the invention relates to a S1P₁ receptor modulator, or a pharmaceutically acceptable salt thereof, for use according to embodiment 35), wherein the threshold value of the IFN-1 gene signature score is −0.5.
37) A further embodiment of the invention relates to a S1P₁ receptor modulator, or a pharmaceutically acceptable salt thereof, for use in a method of preventing or treating (especially treating) a subject having a type 1 interferon mediated disease (especially a type 1 interferon mediated disease that is responsive to treatment with the S1P₁ receptor modulator), the method comprising:
a) providing a biological sample (especially a blood sample or a serum sample, and notably a blood sample) from the subject;
b) assaying gene expression of the type 1 interferon-associated genes RSAD2, IFI27, IFIT1, and HERC5 in the biological sample (especially by measuring peak height in capillary electrophoresis electropherograms);
c) determining a normalised expression value of each assayed gene by (i) measuring the gene expression levels of the three housekeeping (normalizer) genes ACTB, GAPDH, and TFRC in the biological sample of the subject; (ii) calculating the binary logarithm (log₂) of the expression levels of each of the three housekeeping genes and the arithmetic mean thereof; and (iii) calculating the normalized expression value for each of the type 1 interferon-associated genes according to Formula (II)

Normalized Expression Gene i=Log₂ (Expression Level of Gene i)−Mean (Log₂ (Expression Level of Normalizer Genes)) Formula (II);
d) calculating a type 1 interferon gene signature score by averaging the normalised expression values; and
e) treating the subject with the S1P₁ receptor modulator when the type 1 interferon gene signature score of the subject is higher than a threshold value, wherein the threshold value is selected from values between −1.0 and 0 (especially −0.5).

In a preferred embodiment the normalized expression values are calculated based on peak heights of the respective genes by using Formula (I).

38) A further embodiment of the invention relates to a S1P₁ receptor modulator, or a pharmaceutically acceptable salt thereof, for use in the prevention or treatment (especially treatment) of a type 1 interferon mediated disease (especially a type 1 interferon mediated disease that is responsive to treatment with the S1P₁ receptor modulator) in a subject, wherein at least three (especially at least four) type 1 interferon-associated genes are upregulated in the subject (especially in a biological sample from the subject), wherein the type 1 interferon-associated genes are selected from the group consisting of DHX58, EIF2AK2, IRF7, PARP9, SAMD9L, IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6 (especially IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6, and notably EIF2AK2, IFI6, RSAD2, IFI44, IFI44L, IFI27, MX1, IFIT1, HERC5, and ISG15).

The upregulation of type 1 interferon-associated genes in the subject's expression profile may be by any degree relative to that of a sample from a control (which may be from a sample that is not disease tissue of the subject or from a healthy person not afflicted with the type 1 interferon mediated disease) or may be relative to that of genes from the subject whose expression is not changed by the type 1 interferon mediated disease (so called housekeeping genes); especially relative to that of genes from the subject whose expression is not changed by the type 1 interferon mediated disease.

The degree of upregulation may be at least 10%, at least 15%, at least 20%, at least 25%, at least 30%, at least 40%, at least 50%, at least 60%, at least 70%, at least 80%, at least 90%, at least 100%, at least 150%, at least 200%, at least 300%, or more that of the control or control sample.
39) A further embodiment of the invention relates to a S1P₁ receptor modulator, or a pharmaceutically acceptable salt thereof, for use according to embodiment 38), wherein the type 1 interferon-associated genes are upregulated in the subject at treatment onset.

40) A further embodiment of the invention relates to a S1P₁ receptor modulator, or a pharmaceutically acceptable salt thereof, for use according to any one of embodiments 38) or 39), wherein at least three genes of at least one of the following groups of type 1 interferon-associated genes are upregulated in the subject:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) IFI6, RSAD2, IFI44, IFI44L, and IFI27; or
d) IFI6, RSAD2, IFI44, and IFI44L; or
e) IFI6, RSAD2, IFI44L, and IFI27; or
f) IFI6, RSAD2, IFI44, and IFI27; or
g) RSAD2, IFI44, IFI44L, and IFI27; or
h) IFI6, IFI44, IFI44L, and IFI27; or
i) IFI6, MX1, IFIT1, and HERC5; or
j) EIF2AK2, MX1, and IFIT1; or
k) EIF2AK2, IFI44, and IFIT1.
l) EIF2AK2, IFI6, RSAD2, IFI44, IFI44L, IFI27, MX1, IFIT1, HERC5, and ISG15; or
m) RSAD2, IFI27, IFIT1, and HERC5.
41) A further embodiment of the invention relates to a S1P₁ receptor modulator, or a pharmaceutically acceptable salt thereof, for use according to any one of embodiments 38) or 39), wherein at least three genes of at least one of the following groups of type 1 interferon-associated genes are upregulated in the subject:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) RSAD2, IFI44, IFI44L, and IFI27; or
d) EIF2AK2, IFI6, RSAD2, IFI44, IFI44L, IFI27, MX1, IFIT1, HERC5, and ISG15; or
e) RSAD2, IFI27, IFIT1, and HERC5.
42) A further embodiment of the invention relates to a S1P₁ receptor modulator, or a pharmaceutically acceptable salt thereof, for use according to any one of embodiments 38) or 39), wherein at least three genes of at least one of the following groups of type 1 interferon-associated genes are upregulated in the subject:
a) RSAD2, IFI44, IFI44L, and IFI27; or
b) EIF2AK2, IFI6, RSAD2, IFI44, IFI44L, IFI27, MX1, IFIT1, HERC5, and ISG15; or
c) RSAD2, IFI27, IFIT1, and HERC5.
43) A further embodiment of the invention relates to a S1P₁ receptor modulator, or a pharmaceutically acceptable salt thereof, for use according to any one of embodiments 38) or 39), wherein at least three genes of at least one of the following groups of type 1 interferon-associated genes are upregulated in the subject:
a) RSAD2, IFI44, IFI44L, and IFI27; or
b) RSAD2, IFI27, IFIT1, and HERC5.
44) A further embodiment of the invention relates to a S1P₁ receptor modulator, or a pharmaceutically acceptable salt thereof, for use according to any one of embodiments 38) or 39), wherein at least three of the following type 1 interferon-associated genes are upregulated in the subject: EIF2AK2, IFI6, RSAD2, IFI44, IFI44L, IFI27, MX1, IFIT1, HERC5, and ISG15.
45) A further embodiment of the invention relates to a S1P₁ receptor modulator, or a pharmaceutically acceptable salt thereof, for use according to any one of embodiments 38) or 39), wherein at least three of the following type 1 interferon-associated genes are upregulated in the subject: RSAD2, IFI27, IFIT1, and HERC5.
46) A further embodiment of the invention relates to a S1P₁ receptor modulator, or a pharmaceutically acceptable salt thereof, for use in the prevention or treatment (especially treatment) of a type 1 interferon mediated disease in a subject, wherein the concentration/level of interferon-gamma (IFN-γ) in the subject (especially in blood plasma of the subject) is elevated compared to a healthy subject.

For clarity, “a subject, wherein the concentration/level of interferon-gamma (IFN-γ) in the subject (especially in blood plasma of the subject) is elevated compared to a healthy subject” means a subject having, and/or identified as having, and/or diagnosed as having (especially in blood plasma of the subject) a concentration/level of interferon-gamma (IFN-γ) that is elevated compared to a healthy subject.

Especially, “a subject, wherein the concentration/level of interferon-gamma (IFN-γ) in the subject (especially in blood plasma of the subject) is elevated compared to a healthy subject” means a subject having, and/or identified as having, and/or diagnosed as having (especially in blood plasma of the subject) a concentration/level of interferon-gamma (IFN-γ) that is higher than the concentration/level of interferon-gamma (IFN-γ) in 95% of healthy volunteers in a statistically representative group of the healthy volunteers.
47) A further embodiment of the invention relates to a S1P₁ receptor modulator, or a pharmaceutically acceptable salt thereof, for use in the prevention or treatment (especially treatment) of a type 1 interferon mediated disease in a subject, wherein the subject has a concentration/level (especially a concentration/level in blood plasma) of interferon-gamma (IFN-γ) above 9.5 pg/mL.
48) A further embodiment of the invention relates to a S1P₁ receptor modulator, or a pharmaceutically acceptable salt thereof, for use in a method of preventing or treating (especially treating) a subject having a type 1 interferon mediated disease (that is responsive to treatment with the S1P₁ receptor modulator), the method comprising:
a) providing a biological sample (especially a plasma sample) from the subject;
b) measuring the concentration/level of interferon-gamma (IFN-γ) in the biological sample; and
c) treating the subject with the S1P₁ receptor modulator when the concentration/level of interferon-gamma (IFN-γ) is elevated compared to the concentration/level of interferon-gamma (IFN-γ) in a biological sample from a healthy subject and/or is above 9.5 pg/mL.
49) A further embodiment of the invention relates to a S1P₁ receptor modulator, or a pharmaceutically acceptable salt thereof, for use according to any one of embodiments 1) to 48), wherein the concentration/level of interferon-alpha (IFN-α) and/or interferon-gamma (IFN-γ) in the subject (especially in blood plasma of the subject) is/are elevated compared to a healthy subject.
50) A further embodiment of the invention relates to a S1P₁ receptor modulator, or a pharmaceutically acceptable salt thereof, for use according to any one of embodiments 1) to 48), wherein the subject has a concentration/level (especially a concentration/level in blood plasma) of interferon-alpha (IFN-α) above 0.1 pg/mL and/or of interferon-gamma (IFN-γ) above 9.5 pg/mL.
51) A further embodiment of the invention relates to a S1P₁ receptor modulator, or a pharmaceutically acceptable salt thereof, for use in a method according to any one of embodiments 19) to 37), wherein the method further comprises:
a) providing a biological sample (especially a plasma sample) from the subject;
b) measuring the concentration/level of interferon-alpha (IFN-α) and/or interferon-gamma (IFN-γ) in the biological sample; and
c) treating the subject with the S1P₁ receptor modulator when the concentration/level of interferon-alpha (IFN-α) and/or of interferon-gamma (IFN-γ) is elevated compared to the concentration/level in a biological sample from a healthy subject.

For clarity, treatment of the subject with the S1P₁ receptor modulator according to dependent embodiment 51) requires both conditions to be fulfilled: (i) that the type 1 interferon gene signature score of the subject is higher than the respective threshold value and (ii) that the concentration/level of interferon-alpha (IFN-α) and/or of interferon-gamma (IFN-γ) is elevated compared to the concentration/level in a biological sample from a healthy subject.
52) A further embodiment of the invention relates to a S1P₁ receptor modulator, or a pharmaceutically acceptable salt thereof, for use in a method according to any one of embodiments 19) to 37), wherein the method further comprises:
a) providing a biological sample (especially a plasma sample) from the subject;
b) measuring the concentration/level of interferon-alpha (IFN-α) and/or interferon-gamma (IFN-γ) in the biological sample; and
c) treating the subject with the S1P₁ receptor modulator when the concentration/level of interferon-alpha (IFN-α) is above 0.1 pg/mL and/or when the concentration/level of interferon-gamma (IFN-γ) is above 9.5 pg/mL.

For clarity, treatment of the subject with the S1P₁ receptor modulator according to dependent embodiment 52) requires both conditions to be fulfilled: (i) that the type 1 interferon gene signature score of the subject is higher than the respective threshold value and (ii) that the concentration/level of interferon-alpha (IFN-α) is above 0.1 pg/mL and/or that the concentration/level of interferon-gamma (IFN-γ) is above 9.5 pg/mL.
53) A further embodiment of the invention relates to a S1P₁ receptor modulator, or a pharmaceutically acceptable salt thereof, for use according to any one of embodiments 1) to 52), wherein the S1P₁ receptor modulator is selected from fingolimod, ozanimod, siponimod, ponesimod, cenerimod, etrasimod, mocravimod and amiselimod, or a pharmaceutically acceptable salt thereof (especially from ozanimod, siponimod, ponesimod, cenerimod, etrasimod, mocravimod and amiselimod, or a pharmaceutically acceptable salt thereof, and notably from ozanimod, siponimod, ponesimod, cenerimod, or a pharmaceutically acceptable salt thereof).

Fingolimod (also known as FTY720, Gilenya, Tascenso ODT, 2-amino-2-[2-(4-octylphenyl) ethyl]propan-1,3-diol) is a S1P receptor modulator that binds to several S1P receptors. Fingolimod is used as a hydrochloride salt, is approved by the U.S. Food & Drug administration for the treatment of relapsing forms of multiple sclerosis and is available as hard capsules in strength of 0.25 mg and 0.5 mg. Alternatively, Fingolimod is used as a lauryl sulfate salt, is approved by the U.S. Food & Drug administration for the treatment of relapsing forms of multiple sclerosis and is available as orally disintegrating tablets in strength of 0.25 mg and 0.5 mg.

Ozanimod (also known as RPC1063, Zeposia, 5-(3-{(1S)-1-[(2-hydroxyethyl)amino]-2,3-dihydro-1H-inden-4-yl}-1,2,4-oxadiazol-5-yl)-2-[(propan-2-yl)oxy]benzonitrile) is a S1P receptor modulator that selectively binds to S1P₁ and S1P₅ receptors. Ozanimod is used as a hydrochloride salt, is approved by the U.S. Food & Drug administration for the treatment of relapsing forms of multiple sclerosis and moderately to severely active ulcerative colitis and is available as capsules in strengths of 0.23 mg, 0.46 mg, and 0.92 mg (based on the free base and equivalent to 0.25 mg, 0.5 mg, and 1 mg ozanimod hydrochloride salt). The recommended maintenance dosage is 0.92 mg orally once daily.

Siponimod (also known as BAF312, MAYZENT, 1-[[4-[(1E)-1-[[[4-Cyclohexyl-3-(trifluoromethyl)phenyl]methoxy]imino]ethyl]-2-ethylphenyl]methyl]-3-azetidinecarboxylic acid) is a dual S1P₁ and S1P₅ receptor modulator. Siponimod is used as a (2E)-2-butenedioate (2:1 mixture), is approved by the U.S. Food & Drug administration for the treatment of relapsing forms of multiple sclerosis and is available as tablets in strengths of 0.25 mg, 1 mg, and 2 mg. The recommended maintenance dosage is 1 mg (in patients with a CYP2C9*1/*3 or *2/*3 genotype) or 2 mg orally once daily.

Ponesimod (also known as ACT-128800, PONVORY, (2Z,5Z)-5-[3-chloro-4-[(2R)-2,3-dihydroxypropoxy]benzylidene]-3-(2-methylphenyl)-2-(propylimino)-1,3-thiazolidin-4-one) is a selective modulator of the S1P₁, S1P₄, and S1P₅ receptor. Ponesimod is approved by the U.S. Food & Drug administration for the treatment of relapsing forms of multiple sclerosis and is available as tablets in strengths of 2 mg, 3 mg, 4 mg, 5 mg, 6 mg, 7 mg, 8 mg, 9 mg, 10 mg, and 20 mg. The recommended maintenance dosage is 20 mg orally once daily.

Cenerimod (also known as ACT-334441, (S)-3-{4-[5-(2-cyclopentyl-6-methoxy-pyridin-4-yl)-oxadiazol-3-yl]-2-ethyl-6-methyl-phenoxy}-propane-1,2-diol) is a selective S1P₁ receptor modulator. Cenerimod is developed for the treatment of systemic lupus erythematosus and was used in a clinical phase 2b study as tablets in strengths of 0.5 mg, 1 mg, 2 mg, and 4 mg.

Etrasimod (also known as APD334, 2-[(3R)-7-[[4-cyclopentyl-3-(trifluoromethyl)phenyl]methoxy]-1,2,3,4-tetrahydrocyclopenta[b]indol-3-yl]acetic acid) is an oral, once-a-day selective modulator of S1P₁, S1P₄, and S1P₅. Etrasimod is being investigated for a range of immuno-inflammatory diseases including ulcerative colitis, Crohn's Disease, atopic dermatitis, eosinophilic esophagitis, and alopecia areata and is used as tablets in strengths of 2 mg.
54) A further embodiment of the invention relates to a S1P₁ receptor modulator, or a pharmaceutically acceptable salt thereof, for use according to any one of embodiments 1) to 52), wherein the S1P₁ receptor modulator is selected from ozanimod, siponimod, ponesimod, and cenerimod, or a pharmaceutically acceptable salt thereof.
55) A further embodiment of the invention relates to a S1P₁ receptor modulator, or a pharmaceutically acceptable salt thereof, for use according to any one of embodiments 1) to 52), wherein the S1P₁ receptor modulator is cenerimod.
56) A further embodiment of the invention relates to a S1P₁ receptor modulator, or a pharmaceutically acceptable salt thereof, for use according to embodiment 55), wherein cenerimod is comprised in a pharmaceutical dosage form for oral administration (especially in a tablet formulation, notably in a film coated tablet).
57) A further embodiment of the invention relates to a S1P₁ receptor modulator, or a pharmaceutically acceptable salt thereof, for use according to any one of embodiments 55) or 56), wherein cenerimod is comprised in the pharmaceutical dosage form in a unit dose of 4 mg (especially 4.0 mg).

The term “unit dose”, as used herein, refers to the amount of an active pharmaceutical ingredient (especially cenerimod) that is administered and/or is to be administered to a subject in a single dose. As an example, a unit dose is the amount of an active pharmaceutical ingredient (especially cenerimod) in a tablet or capsule (especially a tablet) for oral administration in case a single tablet or capsule is administered and/or is to be administered to the subject per administration.
58) A further embodiment of the invention relates to a S1P₁ receptor modulator, or a pharmaceutically acceptable salt thereof, for use according to any one of embodiments 55) to 57), wherein cenerimod is to be administered and/or is administered to a patient once per day (once daily).
59) A further embodiment of the invention relates to a S1P₁ receptor modulator, or a pharmaceutically acceptable salt thereof, for use according to any one of embodiments 55) to 58), wherein cenerimod is to be administered and/or is administered to a subject for a treatment period of at least 6 months (especially at least 12 months, and notably at least 60 months).

The term “treatment period”, as used herein (especially in relation to cenerimod), refers to the period between the first day of a treatment and the last day of an uninterrupted treatment with an active pharmaceutical ingredient (especially in relation to cenerimod), a pharmaceutical composition and/or a medicament, wherein a treatment is “uninterrupted” if it is in accordance with a regularly repeating dosing scheme such as, for example, once daily. For instance, if cenerimod is administered and/or is to be administered once daily for a treatment period of 6 months, this means that cenerimod is administered and/or is to be administered once at every day of the 6 months period.
60) A further embodiment of the invention relates to a S1P₁ receptor modulator, or a pharmaceutically acceptable salt thereof, for use according to any one of embodiments 55) to 59), wherein cenerimod is to be administered and/or is administered to a subject chronically.

The term “chronical treatment” (or “chronically”) refers to a treatment period starting with the first day of treatment with the respective active pharmaceutical ingredient (especially cenerimod) and ending on the day on which a further continuation of the treatment of the subject with the active pharmaceutical ingredient is no longer feasible or indicated. The treatment of the subject with the active pharmaceutical ingredient might be for instance no longer feasible or indicated for reasons such as side effects of the treatment, changes of the health condition of the subject (requiring for instance a different medication), changes of other relevant circumstances in the life of a subject, death of the subject and the like. Especially, the term “chronical treatment” (or “chronically”) refers to a treatment period of at least 10 years, starting with the first day of treatment with the respective active pharmaceutical ingredient (especially cenerimod) until a further continuation of the treatment of the subject with the active pharmaceutical ingredient is not feasible or indicated.

The term “treatment onset” refers to the first day of treatment with the respective active pharmaceutical ingredient (especially the S1P₁ receptor modulator; and notably cenerimod).

The term “type 1 interferon mediated disease” means a disease that is linked or related to and/or has a common cause with (especially that is linked to and/or has a common cause with) a high type 1 interferon (IFN-1) gene signature score in at least some of the subjects suffering from the disease.
61) A further embodiment of the invention relates to a S1P₁ receptor modulator, or a pharmaceutically acceptable salt thereof, for use according to any one of embodiments 1) to 60), wherein the type 1 interferon mediated disease is selected from the group consisting of systemic lupus erythematosus, discoid lupus, lupus nephritis, glomerulonephritis, type I diabetes, inflammatory bowel disease (including Crohn's disease, ulcerative colitis, and Celiac's disease), multiple sclerosis, autoimmune thyroiditis, scleroderma, psoriasis, primary Sjogren's disease, systemic sclerosis, rheumatoid arthritis, transplant rejection, dermatomyositis, polymyositis, idiopathic inflammatory myositis, sarcoidosis, Aicardi-Goutières syndrome, vasculitis, Sting associated vasculopathy with onset in infancy (SAVI) or chronic atypical neutrophilic dermatosis with lipodystrophy and elevated temperature syndrome (CANDLE).

The S1P₁ receptor modulator (especially cenerimod) may be administered for the treatment of a type 1 interferon mediated disease or prophylactically in order to reduce the risk of developing a type 1 interferon mediated disease and/or to delay the onset of the symptoms.

The term “treat” or “treatment” or “treating” used with reference to a disease means either that said disease is cured in the subject; or that, although the subject remains affected by the disease, part or all of the symptoms of said disease are either reduced or eliminated.

The term “prevent” or “prevention” or “preventing” (or alternatively “prophylaxis” or “prophylactical(ly)”) used with reference to a disease means that the risk of developing said disease in the subject is reduced and/or that the onset of one or more symptoms of said disease in the subject is delayed.
62) A further embodiment of the invention relates to a S1P₁ receptor modulator, or a pharmaceutically acceptable salt thereof, for use according to any one of embodiments 1) to 60), wherein the type 1 interferon mediated disease is systemic lupus erythematosus and/or lupus nephritis.
63) A further embodiment of the invention relates to a S1P₁ receptor modulator, or a pharmaceutically acceptable salt thereof, for use according to any one of embodiments 1) to 60), wherein the type 1 interferon mediated disease is systemic lupus erythematosus.
64) A further embodiment of the invention relates to a S1P₁ receptor modulator, or a pharmaceutically acceptable salt thereof, for use according to any one of embodiments 1) to 60), wherein the type 1 interferon mediated disease is lupus nephritis, cutaneous lupus, or lupus with central nervous system (CNS) manifestations.
65) A further embodiment of the invention relates to a S1P₁ receptor modulator, or a pharmaceutically acceptable salt thereof, for use according to any one of embodiments 1) to 64), wherein a reduction from baseline in the modified systemic lupus erythematosus disease activity index 2000 (mSLEDAI-2K) is reached after 12 months treatment of the subject (especially of a subject having SLE) with the S1P₁ receptor modulator (especially cenerimod).

The mSLEDAI-2K score is based on the SLEDAI-2K (Gladman D D, et al. J Rheumatol 2002; 29(2) 288-291), modified to exclude leukopenia (Hermann V, et al. Lupus Science & Medicine 2019; 6:e000354. doi:10.1136/lupus-2019-000354; ClinicalTrials.gov Identifier: NCT03742037).

The term “baseline”, as used herein in connection with a value, a score, an index, or the like (such as, for instance, a type 1 interferon (IFN-1) gene signature score or mSLEDAI-2K etc.), refers to the value, score, index, or the like as measured, calculated or otherwise determined before (especially closely before and notably immediately before) treatment onset with the active pharmaceutical ingredient (such as the S1P₁ receptor modulator or cenerimod).
66) A further embodiment of the invention relates to a S1P₁ receptor modulator, or a pharmaceutically acceptable salt thereof, for use according to any one of embodiments 1) to 64), wherein a reduction from baseline in the modified systemic lupus erythematosus disease activity index 2000 (mSLEDAI-2K) of at least 2 points is reached after 12 months treatment of the subject (especially of a subject having SLE) with the S1P₁ receptor modulator (especially cenerimod).
67) A further embodiment of the invention relates to a S1P₁ receptor modulator, or a pharmaceutically acceptable salt thereof, for use according to any one of embodiments 1) to 64), wherein a reduction from baseline in the modified systemic lupus erythematosus disease activity index 2000 (mSLEDAI-2K) of at least 4 points is reached after 12 months treatment of the subjects (especially of subjects having SLE) with the S1P₁ receptor modulator (especially cenerimod) in at least 30% of the subjects.
68) A further embodiment of the invention relates to a S1P₁ receptor modulator, or a pharmaceutically acceptable salt thereof, for use according to embodiment 67), wherein the reduction from baseline in mSLEDAI-2K is reached in at least 50% of the subjects.
69) A further embodiment of the invention relates to a S1P₁ receptor modulator, or a pharmaceutically acceptable salt thereof, for use according to embodiment 67), wherein the reduction from baseline in mSLEDAI-2K is reached in at least 70% of the subjects.
70) A further embodiment of the invention relates to a S1P₁ receptor modulator, or a pharmaceutically acceptable salt thereof, for use according to any one of embodiments 1) to 64), wherein the modified systemic lupus erythematosus disease activity index 2000 (mSLEDAI-2K) after 12 months treatment of the subjects (especially of subjects having SLE) with either the S1P₁ receptor modulator (especially cenerimod) or placebo is statistically at least 1 point lower in the S1P₁ receptor modulator treated subjects than in placebo treated subjects.
71) A further embodiment of the invention relates to a S1P₁ receptor modulator, or a pharmaceutically acceptable salt thereof, for use according to any one of embodiments 1) to 64), wherein the modified systemic lupus erythematosus disease activity index 2000 (mSLEDAI-2K) after 12 months treatment of the subjects (especially of subjects having SLE) with either the S1P₁ receptor modulator (especially cenerimod) or placebo is statistically at least 1.5 points lower in the S1P₁ receptor modulator treated subjects than in placebo treated subjects.
72) A further embodiment of the invention relates to a S1P₁ receptor modulator, or a pharmaceutically acceptable salt thereof, for use according to any one of embodiments 1) to 64), wherein the modified systemic lupus erythematosus disease activity index 2000 (mSLEDAI-2K) after 12 months treatment of the subjects (especially of subjects having SLE) with either the S1P₁ receptor modulator (especially cenerimod) or placebo is statistically at least 2 points lower in the S1P₁ receptor modulator treated subjects than in placebo treated subjects.
73) A further embodiment of the invention relates to a S1P₁ receptor modulator, or a pharmaceutically acceptable salt thereof, for use according to any one of embodiments 1) to 72), wherein a response on SRI-4 after 12 months treatment of the subjects (especially of subjects having SLE) with the S1P₁ receptor modulator (especially with cenerimod) is reached in at least 30% of the subjects.
74) A further embodiment of the invention relates to a S1P₁ receptor modulator, or a pharmaceutically acceptable salt thereof, for use according to any one of embodiments 1) to 72), wherein a response on SRI-4 after 12 months treatment of the subjects (especially of subjects having SLE) with the S1P₁ receptor modulator (especially with cenerimod) is reached in at least 50 % of the subjects.
75) A further embodiment of the invention relates to a S1P₁ receptor modulator, or a pharmaceutically acceptable salt thereof, for use according to any one of embodiments 1) to 72), wherein a response on SRI-4 after 12 months treatment of the subjects (especially of subjects having SLE) with the S1P₁ receptor modulator (especially with cenerimod) is reached in at least 70% of the subjects.
76) A further embodiment of the invention relates to a S1P₁ receptor modulator, or a pharmaceutically acceptable salt thereof, for use according to any one of embodiments 1) to 72), wherein the probability of a response on SRI-4 after 12 months treatment of a subject (especially of a subject having SLE) is higher (especially statistically significant higher) in a subject treated with the S1P₁ receptor modulator (especially with cenerimod) than in a subject treated with placebo.

A response on SLE responder index (SRI)-4 is defined as:
reduction of at least 4 points in the mSLEDAI-2K compared to baseline; and
no new BILAG A organ domain score and not more than one new BILAG B organ domain score compared to baseline; and
no worsening from baseline in subjects' lupus disease activity, where worsening is defined by an increase ≥0.30 points on a 3-point Physician's Global Assessment (PGA) visual analogue scale (VAS).

The BILAG (British Isles Lupus Assessment Group) score is defined in Romero-Diaz J et al Arthritis Care Res (Hoboken). 2011 Nov; 63 Suppl 11(0 11): S37-46. doi: 10.1002/acr.20572.

The Physician's Global Assessment (PGA) is a visual analog scale (VAS) using 3 benchmarks for assessing disease activity over the last 2 weeks (Luijten KMAC et al, Autoimmunity Reviews Volume 11, Issue 5, March 2012, Pages 326-329). Mild flare will score 1.0 point, moderate flares will score a 2.0-2.5 point and severe flares will score a 3 on the 0-3 analog scale.
77) A further embodiment of the invention relates to a S1P₁ receptor modulator, or a pharmaceutically acceptable salt thereof, for use according to any one of embodiments 1) to 76), wherein the time to first confirmation of a 4-month sustained mSLEDAI-2K response (defined as a reduction of at least 4 points from baseline) is shorter (especially statistically significant shorter) in subject(s) treated with the S1P₁ receptor modulator (especially with cenerimod) than in subject(s) treated with placebo.
78) A further embodiment of the invention relates to cenerimod, or a pharmaceutically acceptable salt thereof, for use according to any one of embodiments 1) to 76), wherein the time from treatment onset with cenerimod to first confirmation of a 4-month sustained mSLEDAI-2K response (defined as a reduction of at least 4 points from baseline) is statistically significant shorter in subject(s) treated with cenerimod than in subject(s) treated with placebo.
79) A further embodiment of the invention relates to a S1P₁ receptor modulator, or a pharmaceutically acceptable salt thereof, for use according to any one of embodiments 1) to 76), wherein the time to first confirmation of a 4-month sustained response in mucocutaneous manifestations is shorter (especially statistically significant shorter) in subject(s) treated with the S1P₁ receptor modulator (especially with cenerimod) than in subject(s) treated with placebo.

The time to first confirmation of a 4-month sustained response in mucocutaneous manifestations is defined as:
no increase in overall mSLEDAI-2K score; and
remission (score of zero) from baseline in the mSLEDAI-2K score of mucocutaneous manifestations (i.e. rash, alopecia, muscoal ulcers).
80) A further embodiment of the invention relates to cenerimod, or a pharmaceutically acceptable salt thereof, for use according to any one of embodiments 1) to 76), wherein the time from treatment onset with cenerimod to first confirmation of a 4-month sustained response in mucocutaneous manifestations is statistically significant shorter in subject(s) treated with cenerimod than in subject(s) treated with placebo.
81) A further embodiment of the invention relates to a S1P₁ receptor modulator, or a pharmaceutically acceptable salt thereof, for use according to any one of embodiments 1) to 80), wherein the S1P₁ receptor modulator is cenerimod, or a pharmaceutically acceptable salt thereof, and wherein a clinically proven effective amount of cenerimod is administered and/or is to be administered to the subject.
82) A further embodiment of the invention relates to a S1P₁ receptor modulator, or a pharmaceutically acceptable salt thereof, for use according to any one of embodiments 1) to 81), wherein the subject is a human (especially a human patient).

The term “subject” includes any human or nonhuman animal. “Nonhuman animal” includes all vertebrates. Notably, the term “subject” refers to a human, and especially to a human patient.
83) A further embodiment of the invention relates to a S1P₁ receptor modulator, or a pharmaceutically acceptable salt thereof, for use according to any one of embodiments 1) to 81), wherein the subject is a human patient suffering from a type 1 interferon mediated disease.
84) A further embodiment of the invention relates to a S1P₁ receptor modulator, or a pharmaceutically acceptable salt thereof, for use according to any one of embodiments 1) to 81), wherein the subject is a human patient suffering from systemic lupus erythematosus and/or lupus nephritis.
85) A further embodiment of the invention relates to a S1P₁ receptor modulator, or a pharmaceutically acceptable salt thereof, for use according to any one of embodiments 1) to 81), wherein the subject is a human patient suffering from systemic lupus erythematosus.
86) A further embodiment of the invention relates to a S1P₁ receptor modulator, or a pharmaceutically acceptable salt thereof, for use according to any one of embodiments 1) to 81), wherein the subject is a human patient suffering from systemic lupus erythematosus, and wherein the subject has a mSLEDAI-2K score≥6 and a clinical mSLEDAI-2K score≥4 with at least 2 points for musculoskeletal or mucocutaneous manifestations (i.e., myositis, arthritis, rash, alopecia, mucosal ulcers).

The clinical mSLEDAI-2K is the mSLEDAI-2K assessment score without the inclusion of points attributable to hematuria, proteinuria, pyuria, low complement, increased DNA binding, and thrombocytopenia.
87) A further embodiment of the invention relates to a S1P₁ receptor modulator, or a pharmaceutically acceptable salt thereof, for use according to any one of embodiments 1) to 81), wherein the subject is a human patient suffering from systemic lupus erythematosus, and wherein the subject has (i) a mSLEDAI-2K score≥6 and a clinical mSLEDAI-2K score≥4 with at least 2 points for musculoskeletal or mucocutaneous manifestations (i.e., myositis, arthritis, rash, alopecia, mucosal ulcers); (ii) a British Isles Lupus Assessment Group-2004 (BILAG) Grade B in ≥2 organ systems or a BILAG Grade A in ≥1 organ system; and (iii) Physician's Global Assessment (PGA) score≥1.0 on a 0 to 3 VAS.
88) A further embodiment of the invention relates to a S1P₁ receptor modulator (especially cenerimod), or a pharmaceutically acceptable salt thereof, for use according to any one of embodiments 1) to 87), wherein the subject is a subject receiving standard background therapy (wherein it is understood that such standard background therapy is suitable for the treatment of a type 1 interferon mediated disease, especially of systemic lupus erythematosus).

Standard background therapy for the treatment of systemic lupus erythematosus may be an antimalarial, such as hydroxychloroquine, chloroquine, or quinacrine; mycophenolate mofetil or mycophenolic acid; azathioprine; methotrexate; an oral corticosteroid, such as prednisone, prednisolone, hydrocortisone, methylprednisolone, or dexamethasone; belimumab; anifrolumab; or any other drug approved by the U.S. Food and Drug Administration (FDA), European Medicines Agency (EMA) or a corresponding national regulatory agency and/or used as standard of care at the date of treatment onset with the S1P₁ receptor modulator.

Cenerimod can be used as medicament according to this invention, e.g. in the form of pharmaceutical compositions especially for enteral, or for parenteral administration.

Dosage forms suitable for enteral administration may be tablets or capsules (especially tablets) comprising a pharmaceutical composition comprising an efficacious amount of cenerimod, or a pharmaceutically acceptable salt thereof.

For avoidance of any doubt, it is understood that any pharmaceutical composition comprising cenerimod in a pharmaceutically effective amount may additionally comprise further conventional excipients and/or additives, which may be used alone or in combination.

Reference is made to the extensive literature on the subject for pharmaceutically acceptable excipients and procedures mentioned herein, see for example R. C. Rowe, P. J. Seskey, S. C. Owen, Handbook of Pharmaceutical Excipients, 5th edition, Pharmaceutical Press 2006; Remington, The Science and Practice of Pharmacy, 21st Edition (2005), Part 5, “Pharmaceutical Manufacturing” [published by Lippincott Williams & Wilkins].

Any reference to a S1P₁ receptor modulator in this specification is to be understood as referring also to the pharmaceutically acceptable salts of such a S1P₁ receptor modulator. Especially, any reference to cenerimod refers to cenerimod or a pharmaceutically acceptable salt form of cenerimod, preferably to cenerimod in free base form.

The term “pharmaceutically acceptable salt” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH, 2008 and ‘Pharmaceutical Salts and Co-crystals’, Johan Wouters and Luc Quéré (Eds.), RSC Publishing, 2012.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10 °C to Y plus 10 °C, and preferably to an interval extending from Y minus 5 °C to Y plus 5 °C.

Whenever the word “between” is used to describe a numerical range, it is to be understood that the end points of the indicated range are explicitly included in the range.

It is understood that any embodiment relating to a S1P₁ receptor modulator, or a pharmaceutically acceptable salt thereof, for use in the prevention or treatment (especially treatment) of a type 1 interferon mediated disease also relates to the use of a S1P₁ receptor modulator, or a pharmaceutically acceptable salt thereof, for the preparation of a medicament for the prevention or treatment (especially treatment) of a type 1 interferon mediated disease; and to a method of prevention/prophylaxis or treatment (especially treatment) of a type 1 interferon mediated disease, the method comprising administering to a subject in need thereof an effective amount (especially a pharmaceutically effective amount) of a S1P₁ receptor modulator, or a pharmaceutically acceptable salt thereof.

It is further understood that any embodiment relating to a S1P₁ receptor modulator, or a pharmaceutically acceptable salt thereof, for use in a method of preventing or treating (especially treating) a subject having a type 1 interferon mediated disease also relates to the use of a S1P₁ receptor modulator, or a pharmaceutically acceptable salt thereof, for the preparation of a medicament for use in a method of preventing or treating (especially treating) a subject having a type 1 interferon mediated disease; and to a method of preventing or treating (especially treating) a subject having a type 1 interferon mediated disease, the method comprising administering to a subject in need thereof an effective amount (especially a pharmaceutically effective amount) of a S1P₁ receptor modulator, or a pharmaceutically acceptable salt thereof.
89) A further embodiment of the invention relates to a method for the prophylaxis or treatment (especially treatment) of a type 1 interferon mediated disease, the method comprising administering a pharmaceutically effective amount of a S1P₁ receptor modulator, or a pharmaceutically acceptable salt thereof, to a subject in need thereof, wherein the subject has a high type 1 interferon (IFN-1) gene signature score.
90) The method according to embodiment 89), wherein the subject has a high type 1 interferon (IFN-1) gene signature score at treatment onset.
91) The method according to any one of embodiments 89) or 90), wherein the type 1 interferon gene signature score is calculated based on any one of the following groups of type 1 interferon-associated genes:
A) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) IFI6, RSAD2, IFI44, IFI44L, and IFI27; or
d) IFI6, RSAD2, IFI44, and IFI44L; or
e) IFI6, RSAD2, IFI44L, and IFI27; or
f) IFI6, RSAD2, IFI44, and IFI27; or
g) RSAD2, IFI44, IFI44L, and IFI27; or
h) IFI6, IFI44, IFI44L, and IFI27; or
i) IFI6, MX1, IFIT1,
j) EIF2AK2, MX1, and IFIT1; or
k) EIF2AK2, IFI44, and IFIT1.
l) EIF2AK2, IFI6, RSAD2, IFI44, IFI44L, IFI27, MX1, IFIT1, HERC5, and ISG15; or
m) RSAD2, IFI27, IFIT1, and HERC5.
92) The method according to any one of embodiments 89) or 90), wherein the type 1 interferon gene signature score is calculated based on any one of the following groups of type 1 interferon-associated genes:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) RSAD2, IFI44, IFI44L, and IFI27; or
d) EIF2AK2, IFI6, RSAD2, IFI44, IFI44L, IFI27, MX1, IFIT1, HERC5, and ISG15; or
e) RSAD2, IFI27, IFIT1, and HERC5.
93) The method according to any one of embodiments 89) or 90), wherein the type 1 interferon gene signature score is calculated based on any one of the following groups of type 1 interferon-associated genes:
a) RSAD2, IFI44, IFI44L, and IFI27; or
b) EIF2AK2, IFI6, RSAD2, IFI44, IFI44L, IFI27, MX1, IFIT1, HERC5, and ISG15; or
c) RSAD2, IFI27, IFIT1, and HERC5.

94) The method according to any one of embodiments 89) or 90), wherein the type 1 interferon gene signature score is calculated based on any one of the following groups of type 1 interferon-associated genes:
a) RSAD2, IFI44, IFI44L, and IFI27; or
b) RSAD2, IFI27, IFIT1, and HERC5.

95) The method according to any one of embodiments 89) or 90), wherein the type 1 interferon gene signature score is calculated based on the type 1 interferon-associated genes EIF2AK2, IFI6, RSAD2, IFI44, IFI44L, IFI27, MX1, IFIT1, HERC5, and ISG15.

96) The method according to any one of embodiments 89) or 90), wherein the type 1 interferon gene signature score is calculated based on the type 1 interferon-associated genes RSAD2, IFI27, IFIT1, and HERC5.
97) The method according to any one of embodiments 89) to 96), wherein the type 1 interferon gene signature score of the subject is at or above the minimum of the bimodal distribution curve of the type 1 interferon gene signature scores of SLE patients.
98) The method according to embodiment 97), wherein the type 1 interferon gene signature score of the subject is at least 0.5 units, at least 1 unit, at least 2 units or at least 3 units (especially at least 1 unit) above the minimum of the bimodal distribution curve of the type 1 interferon gene signature scores of SLE patients.

99) The method according to any one of embodiments 89) to 96), wherein the subject has a type 1 interferon gene signature score that is higher than the type 1 interferon gene signature score of a (representative) healthy subject.

100) The method according to any one of embodiments 89) to 96), wherein the type 1 interferon gene signature score of the subject is above a threshold value.
101) The method according to embodiment 100), wherein the threshold value is an IFN-1 gene signature score value that is selected from values between 1.8 to 4.0 (i.e. 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0; especially 2.0 to 3.0, and notably 2.0 to 2.5) standard deviations above the arithmetic mean of the IFN-1 gene signature score values from at least 20, at least 50, at least 100, at least 200, at least 300 or (preferably) at least 500 healthy humans.
102) The method according to embodiment 100), wherein the threshold value of the IFN-1 gene signature score is 2 standard deviations above the arithmetic mean value from at least 100, at least 200, at least 300 or (preferably) at least 500 healthy humans.
103) The method according to embodiment 100), wherein the threshold value of the IFN-1 gene signature score is between −1.0 and 0 (especially about −0.5).
104) The method according to embodiment 100), wherein the threshold value of the IFN-1 gene signature score is −0.5.
105) The method according to any one of embodiments 89) to 104), wherein the type 1 interferon gene signature score of the subject is calculated (determined) by a method comprising the following steps:
(i) measuring the gene (RNA) expression levels of the type 1 interferon-associated genes (especially of RSAD2, IFI44, IFI44L, and IFI27; or of RSAD2, IFI27, IFIT1, and HERC5; and notably of RSAD2, IFI27, IFIT1, and HERC5) in a blood sample of a subject (notably by measuring respective peak height by capillary electrophoresis);
(ii) measuring the gene expression levels of the three housekeeping (normalizer) genes ACTB, GAPDH, and TFRC in a blood sample of the subject;
(iii) calculating the binary logarithm (log₂) of the expression levels of each of the three housekeeping genes and the arithmetic mean thereof;
(iv) calculating the normalized expression for each of the type 1 interferon-associated genes according to Formula (I):

Normalized Expression Gene i=Log₂ (Height Gene i)−Mean (Log₂ (Normalizer Genes Height)) Formula (I)
and (v) calculating the type 1 interferon gene signature score by averaging the normalized expression values of the type 1 interferon-associated genes.
106) The method according to any one of embodiments 91) to 104), wherein the type 1 interferon gene signature score of the subject is calculated (determined) by a method comprising the following steps:
(i) measuring the gene (RNA) expression levels of the type 1 interferon-associated genes (especially of RSAD2, IFI44, IFI44L, and IFI27; or of RSAD2, IFI27, IFIT1, and HERC5; and notably of RSAD2, IFI27, IFIT1, and HERC5) in a blood sample of a subject (notably by measuring respective peak height by capillary electrophoresis);
(ii) measuring the gene expression levels of the three housekeeping (normalizer) genes ACTB, GAPDH, and TFRC in a blood sample of the subject;
(iii) calculating the binary logarithm (log₂) of the expression levels of each of the three housekeeping genes and the arithmetic mean thereof;
(iv) calculating the normalized expression for each of the four type 1 interferon-associated genes according to Formula (I):

Normalized Expression Gene i=Log₂ (Height Gene i)−Mean (Log₂ (Normalizer Genes Height)) Formula (I)
and (v) calculating the type 1 interferon gene signature score by averaging the normalized expression values of the four type 1 interferon-associated genes.
107) A further embodiment of the invention relates to a method for the prophylaxis or treatment (especially treatment) of a type 1 interferon mediated disease (especially a type 1 interferon mediated disease that is responsive to treatment with a S1P₁ receptor modulator) in a subject, the method comprising:
a) providing a biological sample (especially a blood sample or a serum sample, and notably a blood sample) from the subject;
b) assaying gene expression of at least three (especially at least four) type 1 interferon-associated genes selected from the group consisting of DHX58, EIF2AK2, IRF7, PARP9, SAMD9L, IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6 (especially IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6, and notably EIF2AK2, IFI6, RSAD2, IFI44, IFI44L, IFI27, MX1, IFIT1, HERC5, and ISG15) in the biological sample;
c) determining a normalised expression value of each assayed gene;
d) calculating a type 1 interferon gene signature score by averaging the normalised expression values; and
e) administering a pharmaceutically effective amount of a S1P₁ receptor modulator, or a pharmaceutically acceptable salt thereof, to the subject in need thereof when the type 1 interferon gene signature score of the subject is higher than a threshold value.
108) The method according to embodiment 107), wherein the subject has a type 1 interferon gene signature score that is higher than a threshold value at treatment onset and/or is higher than the type 1 interferon gene signature score in a biological sample from a healthy subject at treatment onset.
109) The method according to any one of embodiments 107) or 108), wherein the biological sample is a blood sample.
110) The method according to any one of embodiments 107) to 109), wherein one of the following groups of type 1 interferon-associated genes is selected for assaying gene expression:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) IFI6, RSAD2, IFI44, IFI44L, and IFI27; or
d) IFI6, RSAD2, IFI44, and IFI44L; or
e) IFI6, RSAD2, IFI44L, and IFI27; or
f) IFI6, RSAD2, IFI44, and IFI27; or
g) RSAD2, IFI44, IFI44L, and IFI27; or
h) IFI6, IFI44, IFI44L, and IFI27; or
i) IFI6, MX1, IFIT1, and HERC5; or
j) EIF2AK2, MX1, and IFIT1; or
k) EIF2AK2, IFI44, and IFIT1.
l) EIF2AK2, IFI6, RSAD2, IFI44, IFI44L, IFI27, MX1, IFIT1, HERC5, and ISG15; or
m) RSAD2, IFI27, IFIT1, and HERC5.
111) The method according to any one of embodiments 107) to 109), wherein one of the following groups of type 1 interferon-associated genes is selected for assaying gene expression:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) RSAD2, IFI44, IFI44L, and IFI27; or
d) EIF2AK2, IFI6, RSAD2, IFI44, IFI44L, IFI27, MX1, IFIT1, HERC5, and ISG15; or
e) RSAD2, IFI27, IFIT1, and HERC5.
112) The method according to any one of embodiments 107) to 109), wherein one of the following groups of type 1 interferon-associated genes is selected for assaying gene expression:
a) RSAD2, IFI44, IFI44L, and IFI27; or
b) EIF2AK2, IFI6, RSAD2, IFI44, IFI44L, IFI27, MX1, IFIT1, HERC5, and ISG15; or
c) RSAD2, IFI27, IFIT1, and HERC5.
113) The method according to any one of embodiments 107) to 109), wherein one of the following groups of type 1 interferon-associated genes is selected for assaying gene expression:
a) RSAD2, IFI44, IFI44L, and IFI27; or
b) RSAD2, IFI27, IFIT1, and HERC5.
114) The method according to any one of embodiments 107) to 109), wherein the type 1 interferon-associated genes for assaying gene expression are EIF2AK2, IFI6, RSAD2, IFI44, IFI44L, IFI27, MX1, IFIT1, HERC5, and ISG15.
115) The method according to any one of embodiments 107) to 109), wherein the type 1 interferon-associated genes for assaying gene expression are RSAD2, IFI27, IFIT1, and HERC5.
116) The method according to any one of embodiments 107) to 115), wherein the gene expression is assayed by capillary electrophoresis.
117) The method according to any one of embodiments 107) to 115), wherein the gene expression of the type 1 interferon-associated genes is assayed by measuring peak height in capillary electrophoresis electropherograms.
118) The method according to any one of embodiments 107) to 117), wherein the normalized expression values are determined by (i) measuring the gene expression levels of the three housekeeping (normalizer) genes ACTB, GAPDH, and TFRC in a blood sample of the subject; (ii) calculating the binary logarithm (log₂) of the expression levels of each of the three housekeeping genes and the arithmetic mean thereof; and (iii) calculating the normalized expression value for each of the type 1 interferon-associated genes according to Formula (II)

Normalized Expression Gene i=Log₂ (Expression Level of Gene i)−Mean (Log₂ (Expression Level of Normalizer Genes)) Formula (II).
119) The method according to any one of embodiments 107) to 118), wherein the type 1 interferon gene signature score of the subject is at or above the minimum of the bimodal distribution curve of the type 1 interferon gene signature scores of SLE patients.
120) The method according to embodiment 119), wherein the type 1 interferon gene signature score of the subject is at least 0.5 units, at least 1 unit, at least 2 units or at least 3 units (especially at least 1 unit) above the minimum of the bimodal distribution curve of the type 1 interferon gene signature scores of SLE patients.
121) The method according to any one of embodiments 107) to 118), wherein the threshold value is an IFN-1 gene signature score value that is selected from values between 1.8 to 4.0 (i.e. 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0; especially 2.0 to 3.0, and notably 2.0 to 2.5) standard deviations above the arithmetic mean of the IFN-1 gene signature score values from at least 20, at least 50, at least 100, at least 200, at least 300 or (preferably) at least 500 healthy humans.
122) The method according to any one of embodiments 107) to 118), wherein the threshold value of the IFN-1 gene signature score is 2 standard deviations above the arithmetic mean value from at least 100, at least 200, at least 300 or (preferably) at least 500 healthy humans.
123) The method according to any one of embodiments 107) to 118), wherein the threshold value of the IFN-1 gene signature score is selected from values between −1.0 and 0 (especially about −0.5, and notably −0.5).
124) The method according to any one of embodiments 107) to 118), wherein the threshold value of the IFN-1 gene signature score is −0.5.
125) A further embodiment of the invention relates to a method for the prophylaxis or treatment (especially treatment) of a type 1 interferon mediated disease (especially a type 1 interferon mediated disease that is responsive to treatment with a S1P₁ receptor modulator) in a subject, the method comprising:
a) providing a biological sample (especially a blood sample or a serum sample, and notably a blood sample) from the subject;
b) assaying gene expression of the type 1 interferon-associated genes RSAD2, IFI27, IFIT1, and HERC5 in the biological sample (especially by measuring peak height in capillary electrophoresis electropherograms);
c) determining a normalised expression value of each assayed gene by (i) measuring the gene expression levels of the three housekeeping (normalizer) genes ACTB, GAPDH, and TFRC in the biological sample of the subject; (ii) calculating the binary logarithm (log₂) of the expression levels of each of the three housekeeping genes and the arithmetic mean thereof; and (iii) calculating the normalized expression value for each of the type 1 interferon-associated genes according to Formula (II)

Normalized Expression Gene i=Log₂ (Expression Level of Gene i)−Mean (Log₂ (Expression Level of Normalizer Genes)) Formula (II);
d) calculating a type 1 interferon gene signature score by averaging the normalised expression values; and
e) administering a pharmaceutically effective amount of a S1P₁ receptor modulator, or a pharmaceutically acceptable salt thereof, to the subject in need thereof when the type 1 interferon gene signature score of the subject is higher than a threshold value, wherein the threshold value is selected from values between −1.0 and 0 (especially −0.5).

126) A further embodiment of the invention relates to a method for the prophylaxis or treatment (especially treatment) of a type 1 interferon mediated disease (especially a type 1 interferon mediated disease that is responsive to treatment with a S1P₁ receptor modulator) in a subject, the method comprising administering a pharmaceutically effective amount of a S1P₁ receptor modulator, or a pharmaceutically acceptable salt thereof, to the subject in need thereof, wherein at least three (especially at least four) type 1 interferon-associated genes are upregulated in the subject (especially in a biological sample from the subject), wherein the type 1 interferon-associated genes are selected from the group consisting of DHX58, EIF2AK2, IRF7, PARP9, SAMD9L, IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6 (especially IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6, and notably EIF2AK2, IFI6, RSAD2, IFI44, IFI44L, IFI27, MX1, IFIT1, HERC5, and ISG15).
127) The method according to embodiment 126), wherein the type 1 interferon-associated genes are upregulated in the subject at treatment onset.
128) The method according to any one of embodiments 126) or 127), wherein at least three genes of at least one of the following groups of type 1 interferon-associated genes are upregulated in the subject:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) IFI6, RSAD2, IFI44, IFI44L, and IFI27; or
d) IFI6, RSAD2, IFI44, and IFI44L; or
e) IFI6, RSAD2, IFI44L, and IFI27; or
f) IFI6, RSAD2, IFI44, and IFI27; or
g) RSAD2, IFI44, IFI44L, and IFI27; or
h) IFI6, IFI44, IFI44L, and IFI27; or
i) IF16, MX1, IFIT1, and HERC5; or
j) EIF2AK2, MX1, and IFIT1; or
k) EIF2AK2, IFI44, and IFIT1.
l) EIF2AK2, IFI6, RSAD2, IFI44, IFI44L, IFI27, MX1, IFIT1, HERC5, and ISG15; or
m) RSAD2, IFI27, IFIT1, and HERC5.
129) The method according to any one of embodiments 126) or 127), wherein at least three genes of at least one of the following groups of type 1 interferon-associated genes are upregulated in the subject:
a) DHX58, EIF2AK2, IRF7, PARP9, SAMD9L, IFI6, IFI44, IFI44L, HERC5, and PLSCR1; or
b) IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6; or
c) RSAD2, IFI44, IFI44L, and IFI27; or
d) EIF2AK2, IFI6, RSAD2, IFI44, IFI44L, IFI27, MX1, IFIT1, HERC5, and ISG15; or
e) RSAD2, IFI27, IFIT1, and HERC5.
130) The method according to any one of embodiments 126) or 127), wherein at least three genes of at least one of the following groups of type 1 interferon-associated genes are upregulated in the subject:
a) RSAD2, IFI44, IFI44L, and IFI27; or
b) EIF2AK2, IFI6, RSAD2, IFI44, IFI44L, IFI27, MX1, IFIT1, HERC5, and ISG15; or
c) RSAD2, IFI27, IFIT1, and HERC5.
131) The method according to any one of embodiments 126) or 127), wherein at least three genes of at least one of the following groups of type 1 interferon-associated genes are upregulated in the subject:
a) RSAD2, IFI44, IFI44L, and IFI27; or
b) RSAD2, IFI27, IFIT1, and HERC5.
132) The method according to any one of embodiments 126) or 127), wherein at least three of the following type 1 interferon-associated genes are upregulated in the subject: EIF2AK2, IFI6, RSAD2, IFI44, IFI44L, IFI27, MX1, IFIT1, HERC5, and ISG15.
133) The method according to any one of embodiments 126) or 127), wherein at least three of the following type 1 interferon-associated genes are upregulated in the subject: RSAD2, IFI27, IFIT1, and HERC5.
134) A further embodiment of the invention relates to a method for the prophylaxis or treatment (especially treatment) of a type 1 interferon mediated disease (especially a type 1 interferon mediated disease that is responsive to treatment with a S1P₁ receptor modulator) in a subject, the method comprising administering a pharmaceutically effective amount of a S1P₁ receptor modulator, or a pharmaceutically acceptable salt thereof, to the subject in need thereof, wherein the concentration/level of interferon-gamma (IFN-γ) in the subject (especially in blood plasma of the subject) is elevated compared to a healthy subject.
135) A further embodiment of the invention relates to a method for the prophylaxis or treatment (especially treatment) of a type 1 interferon mediated disease (especially a type 1 interferon mediated disease that is responsive to treatment with a S1P₁ receptor modulator) in a subject, the method comprising administering a pharmaceutically effective amount of a S1P₁ receptor modulator, or a pharmaceutically acceptable salt thereof, to the subject in need thereof, wherein the subject has a concentration/level (especially a concentration/level in blood plasma) of interferon-gamma (IFN-γ) above 9.5 pg/mL.
136) A further embodiment of the invention relates to a method for the prophylaxis or treatment (especially treatment) of a type 1 interferon mediated disease (especially a type 1 interferon mediated disease that is responsive to treatment with a S1P₁ receptor modulator) in a subject, the method comprising:
a) providing a biological sample (especially a plasma sample) from the subject;
b) measuring the concentration/level of interferon-gamma (IFN-γ) in the biological sample; and
c) administering a pharmaceutically effective amount of a S1P₁ receptor modulator, or a pharmaceutically acceptable salt thereof, to the subject in need thereof when the concentration/level of interferon-gamma (IFN-γ) is elevated compared to the concentration/level of interferon-gamma (IFN-γ) in a biological sample from a healthy subject and/or is above 9.5 pg/mL.
137) The method according to any one of embodiments 89) to 136), wherein the concentration/level of interferon-alpha (IFN-α) and/or interferon-gamma (IFN-γ) in the subject (especially in blood plasma of the subject) is/are elevated compared to a healthy subject.
138) The method according to any one of embodiments 89) to 136), wherein the subject has a concentration/level (especially a concentration/level in blood plasma) of interferon-alpha (IFN-α) above 0.1 pg/mL and/or of interferon-gamma (IFN-γ) above 9.5 pg/mL.
139) The method according to any one of embodiments 107) to 125), wherein the method further comprises:
a) providing a biological sample (especially a plasma sample) from the subject;
b) measuring the concentration/level of interferon-alpha (IFN-α) and/or interferon-gamma (IFN-γ) in the biological sample; and
c) administering a pharmaceutically effective amount of a S1P₁ receptor modulator, or a pharmaceutically acceptable salt thereof, to the subject in need thereof when in addition to the other condition the concentration/level of interferon-alpha (IFN-α) and/or of interferon-gamma (IFN-γ) is elevated compared to the concentration/level in a biological sample from a healthy subject.
140) The method according to any one of embodiments 107) to 125), wherein the method further comprises:
a) providing a biological sample (especially a plasma sample) from the subject;
b) measuring the concentration/level of interferon-alpha (IFN-α) and/or interferon-gamma (IFN-γ) in the biological sample; and
c) administering a pharmaceutically effective amount of a S1P₁ receptor modulator, or a pharmaceutically acceptable salt thereof, to the subject in need thereof when in addition to the other condition the concentration/level of interferon-alpha (IFN-α) is above 0.1 pg/mL and/or when the concentration/level of interferon-gamma (IFN-γ) is above 9.5 pg/mL.
141) The method according to any one of embodiments 89) to 140), wherein the S1P₁ receptor modulator is selected from fingolimod, ozanimod, siponimod, ponesimod, cenerimod, etrasimod, mocravimod and amiselimod, or a pharmaceutically acceptable salt thereof (especially from ozanimod, siponimod, ponesimod, cenerimod, etrasimod, mocravimod and amiselimod, or a pharmaceutically acceptable salt thereof, and notably from ozanimod, siponimod, ponesimod, cenerimod, or a pharmaceutically acceptable salt thereof).
142) The method according to any one of embodiments 89) to 140), wherein the S1P₁ receptor modulator is selected from ozanimod, siponimod, ponesimod, and cenerimod, or a pharmaceutically acceptable salt thereof.
143) The method according to any one of embodiments 89) to 140), wherein the S1P₁ receptor modulator is cenerimod.
144) The method according to embodiment 143), wherein cenerimod is comprised in a pharmaceutical dosage form for oral administration (especially in a tablet formulation, notably in a film coated tablet).
145) The method according to any one of embodiments 143) or 144), wherein cenerimod is comprised in the pharmaceutical dosage form in a unit dose of 4 mg (especially 4.0 mg).
146) The method according to any one of embodiments 143) to 145), wherein cenerimod is to be administered and/or is administered to a patient once per day (once daily).
147) The method according to any one of embodiments 143) to 146), wherein cenerimod is to be administered and/or is administered to a subject for a treatment period of at least 6 months (especially at least 12 months, and notably at least 60 months).
148) A further embodiment of the invention relates to a S1P₁ receptor modulator, or a pharmaceutically acceptable salt thereof, for use according to any one of embodiments 143) to 147), wherein cenerimod is to be administered and/or is administered to a subject chronically.
149) The method according to any one of embodiments 89) to 148), wherein the type 1 interferon mediated disease is selected from the group consisting of systemic lupus erythematosus, discoid lupus, lupus nephritis, glomerulonephritis, type I diabetes, inflammatory bowel disease (including Crohn's disease, ulcerative colitis, and Celiac's disease), multiple sclerosis, autoimmune thyroiditis, scleroderma, psoriasis, primary Sjogren's disease, systemic sclerosis, rheumatoid arthritis, transplant rejection, dermatomyositis, polymyositis, idiopathic inflammatory myositis, sarcoidosis, Aicardi-Goutières syndrome, vasculitis, Sting associated vasculopathy with onset in infancy (SAVI) or chronic atypical neutrophilic dermatosis with lipodystrophy and elevated temperature syndrome (CANDLE).
150) The method according to any one of embodiments 89) to 148), wherein the type 1 interferon mediated disease is systemic lupus erythematosus and/or lupus nephritis.
151) The method according to any one of embodiments 89) to 148), wherein the type 1 interferon mediated disease is systemic lupus erythematosus.
152) The method according to any one of embodiments 89) to 148), wherein the type 1 interferon mediated disease is lupus nephritis, cutaneous lupus, or lupus with central nervous system (CNS) manifestations.
153) The method according to any one of embodiments 89) to 152), wherein a reduction from baseline in the modified systemic lupus erythematosus disease activity index 2000 (mSLEDAI-2K) is reached after 12 months treatment of the subject (especially of a subject having SLE) with the S1P₁ receptor modulator (especially cenerimod).
154) The method according to any one of embodiments 89) to 152), wherein a reduction from baseline in the modified systemic lupus erythematosus disease activity index 2000 (mSLEDAI-2K) of at least 2 points (especially at least 4 points) is reached after 12 months treatment of the subject (especially of a subject having SLE) with the S1P₁ receptor modulator (especially cenerimod).
155) The method according to any one of embodiments 89) to 152), wherein a reduction from baseline in the modified systemic lupus erythematosus disease activity index 2000 (mSLEDAI-2K) of at least 4 points is reached after 12 months treatment of the subjects (especially of subjects having SLE) with the S1P₁ receptor modulator (especially cenerimod) in at least 30% of the subjects.
156) The method according to any one of embodiments 153) to 155), wherein the reduction from baseline in mSLEDAI-2K is reached in at least 50% of the subjects.
157) The method according to any one of embodiments 153) to 155), wherein the reduction from baseline in mSLEDAI-2K is reached in at least 70% of the subjects.
158) The method according to any one of embodiments 89) to 152), wherein the modified systemic lupus erythematosus disease activity index 2000 (mSLEDAI-2K) after 12 months treatment of the subjects (especially of subjects having SLE) with either the S1P₁ receptor modulator (especially cenerimod) or placebo is statistically at least 1 point lower in the S1P₁ receptor modulator treated subjects than in placebo treated subjects.
159) The method according to any one of embodiments 89) to 152), wherein the modified systemic lupus erythematosus disease activity index 2000 (mSLEDAI-2K) after 12 months treatment of the subjects (especially of subjects having SLE) with either the S1P₁ receptor modulator (especially cenerimod) or placebo is statistically at least 1.5 points lower in the S1P₁ receptor modulator treated subjects than in placebo treated subjects.
160) The method according to any one of embodiments 89) to 152), wherein the modified systemic lupus erythematosus disease activity index 2000 (mSLEDAI-2K) after 12 months treatment of the subjects (especially of subjects having SLE) with either the S1P₁ receptor modulator (especially cenerimod) or placebo is statistically at least 2 points lower in the S1P₁ receptor modulator treated subjects than in placebo treated subjects.

161) The method according to any one of embodiments 89) to 160), wherein a response on SRI-4 after 12 months treatment of the subjects (especially of subjects having SLE) with the S1P₁ receptor modulator (especially with cenerimod) is reached in at least 30% of the subjects.
162) The method according to any one of embodiments 89) to 160), wherein a response on SRI-4 after 12 months treatment of the subjects (especially of subjects having SLE) with the S1P₁ receptor modulator (especially with cenerimod) is reached in at least 50% of the subjects.
163) The method according to any one of embodiments 89) to 160), wherein a response on SRI-4 after 12 months treatment of the subjects (especially of subjects having SLE) with the S1P₁ receptor modulator (especially with cenerimod) is reached in at least 70% of the subjects.
164) The method according to any one of embodiments 89) to 160), wherein the probability of a response on SRI-4 after 12 months treatment of a subject (especially of a subject having SLE) is higher (especially statistically significant higher) in a subject treated with the S1P₁ receptor modulator (especially with cenerimod) than in a subject treated with placebo.
165) The method according to any one of embodiments 89) to 164), wherein the time to first confirmation of a 4-month sustained mSLEDAI-2K response (defined as a reduction of at least 4 points from baseline) is shorter (especially statistically significant shorter) in subject(s) treated with the S1P₁ receptor modulator (especially with cenerimod) than in subject(s) treated with placebo.
166) The method according to any one of embodiments 89) to 164), wherein the S1P₁ receptor modulator is cenerimod and wherein the time from treatment onset with cenerimod to first confirmation of a 4-month sustained mSLEDAI-2K response (defined as a reduction of at least 4 points from baseline) is statistically significant shorter in subject(s) treated with cenerimod than in subject(s) treated with placebo.
167) The method according to any one of embodiments 89) to 164), wherein the time to first confirmation of a 4-month sustained response in mucocutaneous manifestations is shorter (especially statistically significant shorter) in subject(s) treated with the S1P₁ receptor modulator (especially with cenerimod) than in subject(s) treated with placebo.
168) The method according to any one of embodiments 89) to 164), wherein the S1P₁ receptor modulator is cenerimod and wherein the time from treatment onset with cenerimod to first confirmation of a 4-month sustained response in mucocutaneous manifestations is statistically significant shorter in subject(s) treated with cenerimod than in subject(s) treated with placebo.
169) The method according to any one of embodiments 89) to 168), wherein the S1P₁ receptor modulator is cenerimod, or a pharmaceutically acceptable salt thereof, and wherein a clinically proven effective amount of cenerimod is administered and/or is to be administered to the subject.
170) The method according to any one of embodiments 89) to 169), wherein the subject is a human (especially a human patient).
171) The method according to any one of embodiments 89) to 169), wherein the subject is a human patient suffering from a type 1 interferon mediated disease.
172) The method according to any one of embodiments 89) to 169), wherein the subject is a human patient suffering from systemic lupus erythematosus and/or lupus nephritis.
173) The method according to any one of embodiments 89) to 169), wherein the subject is a human patient suffering from systemic lupus erythematosus.
174) The method according to any one of embodiments 89) to 169), wherein the subject is a human patient suffering from systemic lupus erythematosus, and wherein the subject has a mSLEDAI-2K score≥6 and a clinical mSLEDAI-2K score≥4 with at least 2 points for musculoskeletal or mucocutaneous manifestations (i.e., myositis, arthritis, rash, alopecia, mucosal ulcers).
175) The method according to any one of embodiments 89) to 169), wherein the subject is a human patient suffering from systemic lupus erythematosus, and wherein the subject has (i) a mSLEDAI-2K score≥6 and a clinical mSLEDAI-2K score≥4 with at least 2 points for musculoskeletal or mucocutaneous manifestations (i.e., myositis, arthritis, rash, alopecia, mucosal ulcers); (ii) a British Isles Lupus Assessment Group-2004 (BILAG) Grade B in ≥2 organ systems or a BILAG Grade A in ≥1 organ system; and (iii) Physician's Global Assessment (PGA) score≥1.0 on a 0 to 3 VAS.
176) The method according to any one of embodiments 89) to 175), wherein the subject is a subject receiving standard background therapy (wherein it is understood that such standard background therapy is suitable for the treatment of a type 1 interferon mediated disease, especially of systemic lupus erythematosus).
177) A further embodiment of the invention relates to a method of treating a type 1 interferon mediated disease (especially systemic lupus erythematosus) in a subject in need thereof, wherein the subject has a high type 1 interferon (IFN-1) gene signature score; the method comprising administering to the subject a pharmaceutical composition comprising a clinically proven effective amount of a S1P₁ receptor modulator (especially cenerimod), or a pharmaceutically acceptable salt thereof.
178) The method according to embodiment 177), wherein the S1P₁ receptor modulator is cenerimod and wherein said clinically proven effective amount of cenerimod is 4.0 mg per day.
179) The method according to any one of embodiments 177) or 178), wherein said method comprises administering to the subject a pharmaceutical composition comprising a clinically proven safe and clinically proven effective amount of the S1P₁ receptor modulator (especially cenerimod).

As used herein, unless otherwise noted, the term “clinically proven” (used independently or to modify the terms “safe” and/or “effective”) shall mean that it has been proven by a clinical trial wherein the clinical trial has met the approval standards of U.S. Food and Drug Administration (FDA), European Medicines Agency (EMA) or a corresponding national regulatory agency. For example, the clinical study may be an adequately sized, randomized, double-blinded study used to clinically prove the effects of the drug.

The term “clinically proven effective” as used herein in the context of a dose, dosage regimen, treatment or method refers to the effectiveness of a particular dose, dosage or treatment regimen. Efficacy can be measured based on change in the course of the disease in response to an agent of the present invention. For example, a S1P₁ receptor modulator (especially cenerimod) is administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life or patient-reported outcome questionnaires developed for a given disease. For example, a compound of the present invention can be administered to achieve an improvement in a subject's condition related to systemic lupus erythematosus. Improvement can be indicated by an improvement in an index of disease activity, by amelioration of clinical symptoms or by any other measure of disease activity.

In a particular embodiment, such measure of disease activity is the modified systemic lupus erythematosus disease activity index 2000 as compared to baseline and/or as compared to placebo.

In another particular embodiment, such measure of disease is the Systemic Lupus Erythematosus Responder Index (SRI-4) as compared to baseline and/or as compared to placebo.

In another particular embodiment, such measure of disease is the time to first confirmation of a 4-month sustained mSLEDAI-2K response (defined as a reduction of at least 4 points from baseline) as compared to placebo.

In another particular embodiment, such measure of disease is the time to first confirmation of a 4-month sustained response in mucocutaneous manifestations as compared to placebo.

In certain embodiments, a clinically proven effective amount of cenerimod is an amount that results in a reduction in the modified systemic lupus erythematosus disease activity index 2000 as compared to baseline and/or as compared to placebo; as demonstrated via clinical trial having the protocol as described in the “Opus Study Overview” herein below.

The term “clinically proven safe,” as it relates to a dose, dosage regimen, treatment or method with the compound of the present invention, cenerimod, refers to a favorable risk: benefit ratio with an acceptable frequency and/or acceptable severity of treatment-emergent adverse events (referred to as AEs or TEAEs). As used herein, “adverse event”, “treatment-emergent adverse event”, and “adverse reaction” mean any harm, unfavorable, unintended, or undesired sign or outcome associated with or caused by administration of a pharmaceutical composition or therapeutic. It is an untoward medical occurrence in a subject administered a medicinal product.

### ABBREVIATIONS

BICLA British Isles Lupus Assessment Group-based Composite Lupus Assessment
BILAG British Isles Lupus Assessment Group
CT Computed tomography
DNA Deoxyribonucleic acid
ECG Electrocardiogram
EDTA Ethylenediaminetetraacetic acid
FDA U.S. Food and Drug Administration
ICF Informed Consent Form
IFN Interferon
IFNAR Interferon alpha receptor
IFNGR Interferon gamma receptor
i.v. intravenous(ly)
JAK Janus kinase
(m)SLEDAI-2K (modified) Systemic lupus erythematosus disease activity index 2000
NSAID Non-steroidal anti-inflammatory drug(s)
(O)CS (oral) Corticosteroid(s)
PCR Polymerase chain reaction
PD (response) Pharmacodynamic (response)
PGA (score) Physician's Global Assessment (score)
RFU Relative Fluorescence Unit(s)
RNA Ribonucleic acid
SLE Systemic lupus erythematosus
SRI Systemic Lupus Erythematosus Responder Index
SRI-4 SRI with an improvement from baseline of at least 4 points in the
mSLEDAI-2K
STAT Signal transducer and activator of transcription
TYK Tyrosine kinase
ULN Upper Limit of Normal
VAS Visual analogue scale (VAS).

### Experimental Part

In the Phase 2b clinical study (CARE), the placebo cohort and cenerimod 4 mg cohort distribution of the IFN-1 gene expression signature score was measured (FIG. 1A). A bimodal distribution was observed and a cut-off of −0.5 was used to separate IFN high from IFN low patients. Surprisingly, there was a strong reduction in the IFN score after 6 months treatment with cenerimod 4 mg compared to placebo (FIG. 1B). Upon subgroup analysis of patients categorized as IFN high and IFN low at baseline unexpectedly, only a reduction in mSLEDAI-2K to baseline was observed in the IFN high subgroup. (FIG. 1C). In the IFN-1 high patient subgroup, a larger change from baseline in mSLEDAI-2K was observed in the 4 mg group compared to placebo with a least square LS-Means (SE) change from baseline to month 6 of −2.6 for placebo and −5.4 for cenerimod 4 mg (FIG. 1C). This improvement in mSLEDAI-2K in IFN-1 high patients was seen even though reductions in blood lymphocyte counts were observed to an equal extend both in IFN-1 low and high patients (FIG. 2), suggesting that lymphocyte reduction is not predictive of mSLEDAI-2K reduction.

Further cenerimod reduces both the IFN-1 gene signature score and IFN-α protein in the plasma of patients treated with cenerimod (FIG. 3A) both of which are more abundant in patients with SLE compared to healthy volunteers (FIG. 3B). Patients with high IFN-α protein levels at baseline display a larger reduction in the mSLEDAI-2K score as compared to the IFN alpha protein low cohort (FIG. 3C). In addition, cenerimod 4 mg reduces IFN gamma protein in plasma at 6 months compared to baseline and compared to placebo (FIG. 4A), which also is more abundant in patients with SLE compared to healthy volunteers (FIG. 4B). Patients with high IFN gamma protein levels at baseline display a larger reduction in the mSLEDAI-2K score as compared to the IFN gamma protein low cohort (FIG. 4C).

Importantly the IFN-1 gene signature score correlated with the measured IFN alpha plasma protein values (FIG. 5A) at baseline strengthening the validity of the IFN gene signature score as a readout of IFN alpha protein. Additionally, cenerimod 4 mg leads to a reduction in the IFN-1 score in both IFN-1 high and IFN-1 low patients at 6 months compared to baseline which is not observed in placebo (FIG. 5B). Surprisingly, despite cenerimod 4 mg lowering the IFN score in all patients equally, the reduction on mSLEDAI-2K is only apparent in IFN-1 high patients.

### Gene Expression Signature

A modulation of biological activity is reflected in changes of gene expression. These changes can be described by specific gene expression signatures. A gene signature is defined by a set of genes that show increased or decreased expression upon the biological activity. Gene signatures are most often analyzed using quantitative reverse transcription PCR (RT-qPCR), which allows standardized expression levels for each gene of interest to be derived.

The IFN-1 signature reflects IFN-regulated genes, meaning that those genes are upregulated due to the biological activity of type 1 IFNs. An IFN-1 gene signature was first described in 2003 in cells isolated from patients with SLE [Baechler, E C. et al., PNAS 2003, 100(5), 2610-2615, PMID:12604793].

An important property of the IFN-1 gene signature is the bimodal distribution in patients with SLE allowing separation into high or low IFN-1 gene signature strata [El-Sherbiny, Y M. et al., Scientific Reports 2018, 8, 5793, DOI:10.1038/s41598-018-24198-1, PMID: 29643425]. There are two most used IFN-1 gene signatures in clinical studies (AstraZeneca [AZ] and DxTerity signature). Although both signatures comprise 4 genes, the difference is in the selection of genes. Both signatures contain IFI27 and RSAD2, but AZ includes in addition IFI44 and IFI44L, whereas DxTerity uses IFIT1 and HERC5. Importantly, the two IFN-1 gene signatures correlate very well and will therefore classify the SLE patients in the same high and low strata as long as the cut-off is derived in the same way. It was previously shown different IFN-1 gene signatures will classify patients into the same IFN-1 status (Abedi M, Comparison Of Different Type 1 IFN Signatures Demonstrates Concordance In A Real World, Home Monitored Systemic Lupus Erythematosus Cohort, EULAR 2018, poster #SAT0041).

The DxTerity gene expression assay contains ten interferon genes (EIF2AK2, HERC5, IFI27, IFI44, IFI44L, IFI6, IFIT1, ISG15, MX1, RSAD2). Four of those genes (HERC5, IFIT1, IFI27, and RSAD2) are used in the IFN-1 test to define the IFN-1 gene signature status (high/low). The assay is using a proprietary Chemical Ligation-dependent Probe Amplification (CLPA/DxDirect) technology.

The DxTerity signature cut-off point of −0.5 (for differentiating IFN high and low subjects) is based on the 95th percentile of more than 500 healthy human (FIG. 1A), while the AZ cut-off point would lead to more than 10% of healthy subjects being classified as high.

Each target gene expression level is normalized to three housekeeping (normalizer) genes (ACTB, GAPDH, and TFRC). The gene expression level of each individual subject is calculated from the capillary electrophoresis electropherogram using the intensity of the fragment peak height (RFU) corresponding to the 4 interferon responsive genes relative to the average of the intensities of three normalizer gene peak heights (from the same subject) using the following formula:

Normalized Expression Gene i=Log2 (Height Gene i)−Mean (Log2 (Normalizer Genes Height))

The IFN-1 gene signature score of the subject is determined by averaging the normalized expression values of the HERC5, IFI27, IFIT1, and RSAD2 genes.

The high/low cut-off (−0.5) is based on the 95th percentile of more than 500 healthy human and is placed 2 standard deviations above the mean IFN-1 gene signature score of the healthy volunteers, and it falls in the trough of the classic bimodal IFN-1 gene signature score distribution seen for SLE patients.

### Plasma Proteins

Interferon alpha (IFNa or IFN-α) was measured in EDTA plasma using an ELISA assay from PBL assay science (41115) following the instructions provided by the supplier. Normal IFNa levels are below the detection limit of the assay (0.1 pg/ml). The high/low cut-off is based on the 95th percentile of 10 healthy human volunteers (FIG. 3B).

Interferon gamma (IFNg or IFN-γ) was measured in EDTA plasma using a V-plex assay from Mesoscale discovery (K151A9H-2) following the instructions provided by the supplier. Normal IFNg levels are reported to be 3.80 pg/ml (median, n=27, 0.46-22.8 pg/ml). As comparison, in-house measurement of 10 EDTA plasma samples from healthy volunteers showed median levels of 1.49 pg/ml (0.49-12.28 pg/ml). The high/low cut-off is based on the 95th percentile of 10 healthy human volunteers (FIG. 4B).

### CARE Study Overview

The study design was a double-blind randomized placebo-controlled study in patients with SLE. A total of 85 patients were included in the 4 mg cenerimod arm and 86 patients in the placebo arm.

At the time of drug initiation 4 mg cenerimod and placebo, defined as baseline, 12.5ml of blood were taken via standard venipuncture by the study nurse and collected in PAX-gene tubes for the subsequent assessment of the IFN gene signature. Tubes containing blood were frozen and remained frozen until further use. EDTA plasma was prepared from blood by standard protocol.

At baseline 36 patients were classified as IFN-1 high and 44 as IFN-1 low in the 4 mg cenerimod arm. In the placebo arm 40 patients were classified as IFN-1 high and 40 patients as IFN-1 low.

The Study design and key inclusion criteria were the following:

The primary objective of the study was to assess the efficacy of 6 months of cenerimod at 4 mg in subjects with moderate to severe SLE. Secondary and exploratory objectives (6 months) included, but not limited, were to the effect on SLE biomarkers and exploratory biomarkers, to assess safety and tolerability of cenerimod doses, to assess the effect on quality of life and fatigue using patient reported outcome (PRO) instrument and to assess the effect on SLE biomarkers.

The inclusion criteria were as follows: A Diagnosis of SLE made at least 6 months prior to Screening (at least 4 ACR criteria), a modified SLEDAI-2K (mSLEDAI-2K) score≥6 of at least 2 points for musculoskeletal or mucocutaneous manifestations (i.e., myositis, arthritis, rash, alopecia, mucosal ulcers) at screening. The modified SLEDAI-2K is defined as SLEDAI-2K with the removal of leukopenia which is one of the scores utilized in the SLEDAI-2K composite score. SLEDAI-2K is described elsewhere (Gladman et al, J Rheumatol. 2002 Feb; 29(2): 288-91). A clinical mSLEDAI-2K score≥4 at randomization was required. Further, a positive Antinuclear Antibody test (ANA) with titre≥1:80 (by immunofluorescence assay) AND/OR positive anti-dsDNA with titre≥30 IU/mL at randomization was required. Participants had to be on a stable background SLE medication for a period of at least 30 days (15 days for corticosteroids) prior to randomization. Participants if using Corticosteroids had to have ≤40 mg/day prednisone or equivalent. Women of childbearing potential (WOCBP) must have agreed to use highly effective methods of contraception.

The exclusion criteria were the following: Patients having active lupus nephritis (defined by proteinuria>1.5 g/24 h, or equivalent using spot urine protein-to-creatinine ratio) or a renal biopsy demonstrating immune complex-mediated glomerulonephritis compatible with lupus nephritis. Patients exhibiting CNS lupus (e.g., aseptic meningitis, seizures, cerebritis, polyneuropathy, cerebrovascular disease, organic brain syndrome) and severe forms of vasculitis requiring systemic immunosuppressive treatment (e.g., retinal vasculitis, coronary vasculitis, pulmonary vasculitis, mesenteric vasculitis) within 90 days prior to screening. Patients presenting with a diagnosis of mixed connective tissue disease or any history of overlap syndromes of SLE with rheumatoid arthritis, erosive arthritis, scleroderma, or autoimmune hepatitis.

The Study treatment was the following: A daily tablet of cenerimod or placebo was taken orally irrespective of food intake and preferably in the morning.

Background therapy criteria: Subjects received at least one of the below listed SLE background medication and had to be stable 30 days prior to randomization except for Oral Corticosteroids (OCS) (which should be stable for at least 15 days prior randomization) NSAIDs: Aspirin (acetylsalicylic acid), Ibuprofen, Naproxen, Celecoxib, Other NSAIDs, Prednisone or equivalent≤40 mg/day, anti-malaria's: Hydroxychloroquine, Chloroquine, Quinacrine, Mycophenolate mofetil, Mycophenolic acid, Azathioprine, Methotrexate, Belimumab must have started at least 30 days prior to Screening. After Randomization, background therapy was kept stable throughout the study. However, if clinically relevant, changes were allowed as follows: Chronic NSAID therapy not to be started or stopped during the study but temporary use and/or dose change for treatment of non-SLE-related conditions (e.g., headache, menstrual cramps) was allowed. Further, immunosuppressant therapy (i.e., methotrexate, azathioprine, mycophenolate mofetil, mycophenolic acid, belimumab) was not started or stopped during the study and the dose was kept stable. Atropine was used (i.v.) in the event of symptomatic bradycardia.

The primary efficacy endpoint is the change from baseline to Month 6 in the modified SLEDAI-2K score. Further secondary endpoints were:

Response on SRI-4 as defined previously (Furie R: et al Arthritis Rheum. 2009 Sep. 15; 61(9):1143-51) at Month 6 as compared to baseline, defined as follows:
Reduction from baseline of at least 4 points in the mSLEDAI-2K; AND
No new organ system affected as defined by one or more BILAG A or 2 or more BILAG B as defined previously (Romero-Diaz J et al Arthritis Care Res (Hoboken). 2011 Nov; 63 Suppl 11(0 11):S37-46. doi:10.1002/acr.20572.) items as compared to baseline; AND
No increase of more than 0.3 points on the Physician's Global Assessment (PGA) since baseline. The Physician Global Assessment (PGA) is a visual analog scale (VAS) using 3 benchmarks for assessing disease activity over the last 2 weeks (Luijten K M A C et al, Autoimmunity Reviews Volume 11, Issue 5, March 2012, Pages 326-329). Mild flare will score 1.0 point, moderate flares will score a 2.0-2.5 point and severe flares will score a 3 on the 0-3 analog scale.

Further, the percent of subjects with no new organ system affected as defined by one or more BILAG A or 2 or more BILAG B items as compared with baseline.

### Opus Study Overview

The Opus studies are two phase 3, multicenter, randomized, double-blind, placebo-controlled, parallel-group study to evaluate the efficacy, safety, and tolerability of cenerimod in adult subjects (18 years to 75 years) with moderate-to-severe systemic lupus erythematosus (SLE) on top of background therapy. Subjects are randomized 1:1 to either cenerimod or matching placebo. Cenerimod is supplied once daily in addition to background SLE therapy as film-coated tablets at the dose of 4 mg.

### Primary Outcome Measures

1. Change from baseline to Month 12 in the modified Systemic Lupus Erythematosus Disease Activity Index-2000 (mSLEDAI-2K) score (Time Frame: Day 1 (pre-dose baseline) to Month 12).
   This endpoint is based on the SLEDAI-2K index, modified to exclude leukopenia. All values of mSLEDAI-2K from baseline through Month 12 visits are accounted for in the assessment of this endpoint.

### Secondary Outcome Measures

1. Response on Systemic Lupus Erythematosus Responder Index (SRI-4) at Month 12 (Time Frame: Day 1 (pre-dose baseline) to Month 12).
   Response on SRI-4 is defined as:
      Reduction from baseline of at least 4 points in the mSLEDAI-2K, and
      No new British Isles Lupus Assessment Group-2004 (BILAG) A organ domain score and not more than one new BILAG B organ domain score compared to baseline, and
      No worsening from baseline in subjects' lupus disease activity, where worsening is defined as an increase ≥0.30 points on a 3-point Physician's Global Assessment visual analog scale, and
      No violation of protocol-specified medication rules detailed in the core protocol.
2. Time to first confirmation of a 4-month sustained modified Systemic Lupus Erythematosus Disease Activity Index-2000 (mSLEDAI-2K) response (Time Frame: Day 1 (pre-dose baseline) to Month 12).
   A response is defined as a reduction of at least 4 points from baseline.
3. Time to first confirmation of a 4-month sustained response in mucocutaneous manifestations (Time Frame: Day 1 (pre-dose baseline) to Month 12).
   Response is defined as:
      No increase in the overall mSLEDAI-2K score, and
      Remission (score of zero) from baseline in the mSLEDAI-2K score of mucocutaneous manifestations.

### Inclusion Criteria

### Inclusion Criteria at Screening

Signed Informed Consent Form (ICF) prior to any study-mandated procedure.
Diagnosis of Systemic Lupus Erythematosus (SLE) made at least 6 months prior to Screening, according to 2019 European League Against Rheumatism/American College of Rheumatology Criteria.
An modified Systemic Lupus Erythematosus Disease Activity Index-2000 (mSLEDAI-2K) score≥6 and clinical mSLEDAI-2K score≥4 with at least 2 points for musculoskeletal or mucocutaneous manifestations (i.e., myositis, arthritis, rash, alopecia, mucosal ulcers). The mSLEDAI-2K score does not include “leukopenia”.
British Isles Lupus Assessment Group-2004 (BILAG) Grade B in ≥2 organ systems or a BILAG Grade A in ≥1 organ system.
Physician's Global Assessment (PGA) score≥1.0 on a 0 to 3 Visual Analogue Scale (VAS).
Currently treated with one or more of the following SLE background medications:
   Anti-malarials (≤400 mg/day hydroxychloroquine, ≤500 mg/day chloroquine, ≤100 mg/day quinacrine).
   Mycophenolate mofetil (≤2 g/day)/mycophenolic acid (≤1.44 g/day).
   Azathioprine (≤2 mg/kg/day).
   Methotrexate (≤25 mg/week).
   Oral Corticosteroids (OCS):
      if OCS is the only SLE background medication: ≥7.5 mg/day and ≤30 mg/day prednisone or equivalent.
      if OCS is not the only SLE background medication: ≤30 mg/day prednisone or equivalent.
   Belimumab (≤10 mg/kg every 4 weeks intravenously, or 200 mg/week subcutaneously (s.c.).
Treatment with antimalarials, mycophenolate mofetil, mycophenolic acid, azathioprine, methotrexate or belimumab must have been started at least 90 days prior to Screening. Treatment with OCS must have been started at least 30 days prior to Screening.
For women of childbearing potential (WoCBP):
   Negative serum pregnancy test at Screening.
   Agreement to undertake monthly urine pregnancy tests from Randomization up to 6 months after study treatment discontinuation.
   Agreement to use a highly effective method of contraception from Screening (Visit 1) up to 6 months after study treatment discontinuation.

### Inclusion Criteria at Randomization

A clinical mSLEDAI-2K score≥4 with at least 2 points for musculoskeletal or mucocutaneous manifestations (i.e., myositis, arthritis, rash, alopecia, mucosal ulcers).
British Isles Lupus Assessment Group-2004 (BILAG) Grade B in 2 or more organ systems or a BILAG Grade A in 1 or more organ system.
Physician's Global Assessment (PGA) score≥1.0 on a 0 to 3 visual analog scale.
Presence of at least one of the following items of serological evidence of active SLE or biological variables predictive of Type 1 Interferon (IFN-1) high signature (in a Screening sample as measured by central laboratory):
   Anti-dsDNA antibodies elevated to above normal.
   Complement C3<lower limit of normal.
   Antinuclear Antibodies with a titer of at least 1:160.
   Anti-Smith antibody elevated to above normal.
   Platelets<200 000/μL.
   Urine protein/creatinine ratio>12.5 mg/mmol (110.5 mg/g).
Currently treated with one or more of the following SLE background medications that must be stable for at least 30 days prior to Randomization (except OCS, which must be stable for at least 15 days prior to Randomization):
   Antimalarials (≤400 mg/day hydroxychloroquine, ≤500 mg/day chloroquine, ≤100 mg/day quinacrine).
   Mycophenolate mofetil (≤2 g/day)/mycophenolic acid (≤1.44 g/day).
   Azathioprine (≤2 mg/kg/day).
   Methotrexate (≤25 mg/week).
   OCS:
      if OCS is the only SLE background medication: ≥7.5 mg/day and ≤30 mg/day prednisone or equivalent.
      if OCS is not the only SLE background medication: ≤30 mg/day prednisone or equivalent).
   Belimumab (≤10 mg/kg every 4 weeks intravenous (i.v.) or ≤200 mg/week s.c.).
WoCBP must have a negative urine pregnancy test at Randomization.

### Main Exclusion Criteria

Pregnant, planning to be become pregnant up to Final Study Visit or lactating women.
Severe central nervous system lupus or active severe or unstable neuropsychiatric SLE characterized by: aseptic meningitis; cerebral vasculitis; myelopathy; demyelination syndromes (ascending, transverse, acute inflammatory demyelinating polyradiculopathy); acute confusional state; impaired level of consciousness; psychosis; acute stroke or stroke syndrome; cranial neuropathy; status epilepticus; cerebellar ataxia; or mononeuritis multiplex:
   That would make the subject unable to fully understand the ICF; OR
   Where, in the opinion of the Principal Investigator, protocol-specified standard of care is insufficient and the use of a more aggressive therapeutic approach, such as adding i.v. cyclophosphamide and/or high dose i.v. pulse corticosteroid (CS) therapy or other treatments not permitted in the protocol is indicated.
A diagnosis of mixed connective tissue disease or any history of overlap syndromes of SLE with psoriasis, rheumatoid arthritis, erosive arthritis, scleroderma, autoimmune hepatitis, or uncontrolled autoimmune thyroid disease.
History or presence of Mobitz type II or third-degree atrioventricular block, sick sinus syndrome, symptomatic bradycardia or syncope associated with cardiac disorders.
Subjects who experienced myocardial infarction, unstable angina pectoris, stroke, transient ischemic attack, vascular thrombosis, decompensated heart failure requiring hospitalization, or heart failure defined by the New York Heart Association Class III/IV within 6 months prior to Screening.
Resting Heart Rate<50 bpm as measured by the 12-lead ECG at Screening or at Randomization.
An elevated QT interval corrected according to Fridericia's formula (QTcF) interval of >470 ms (females)/>450 ms (males) at Screening or at Randomization.
History or presence of severe respiratory disease or pulmonary fibrosis, based on medical history and chest X-ray (or CT scan as per local guidelines), performed at Screening or within 6 months prior to Screening.
History of clinically relevant bronchial asthma or chronic obstructive pulmonary disease that has required treatment with oral or parenteral CS for more than a total of 2 weeks within the last 6 months prior to Screening.
History or presence of malignancy (except for surgically excised basal or squamous cell skin or mucosal lesions, including dysplasia and carcinoma in situ), lymphoproliferative disease, or history of total lymphoid irradiation.
Presence of macular edema or active uveitis detected by optical coherence tomography (OCT) during screening.
History of chronic liver or biliary disease (other than Gilbert's Syndrome) or subjects with alanine aminotransferase or aspartate aminotransferase>3×Upper Limit of Normal (ULN) or total bilirubin>1.5 ULN (unless in the context of known Gilbert's Syndrome).
Significant hematology abnormality at screening assessment:
   lymphocyte count<500/μL (0.5×10\hat{ }9/L);
   hemoglobin<7 g/dL;
   white blood cell count<2000/μL (2.0×10\hat{ }9/L); or
   platelets<25000/μL (25×10\hat{ }9/L) at screening assessment.
Treatment with the following medications within 15 days or 5 half-lives of the medication (whichever is longer) prior to Randomization:
   β-blockers, diltiazem, verapamil, digoxin, digitoxin, or any other antiarrhythmic or heart-rate-lowering systemic therapy.
   QT-prolonging drugs with known risk of torsade de pointes irrespective of indication.
Treatment with the following medications within 30 days or 5 half-lives of the medication (whichever is longer) prior to Randomization:
   Cyclophosphamide, cyclosporine, tacrolimus, sirolimus, mizoribine, etc.
   Pulse methylprednisolone.
   Vaccination with live vaccines (including live vaccines for COVID-19).
Intra-articular, intramuscular or i.v. CS within 6 weeks prior to Randomization.
Treatment with the following medications within 90 days or 5 half-lives of the medication (whichever is longer) prior to Randomization:
   Leflunomide.
   i.v. immunoglobulins.

Treatment with any investigational agent within 90 days or 5 half-lives of the drug (whichever is longer) prior to Randomization.

Treatment with B cell-depleting biological agents, e.g., rituximab or ocrelizumab, within 12 months prior to Randomization.
Treatment with anifrolumab within 12 months prior to Randomization.
Treatment with any of the following medications any time prior to Screening:
   Alemtuzumab.
   Sphingosine-1-phosphate receptor modulators (e.g., fingolimod).
   Subjects previously randomized to cenerimod or placebo in any trial involving cenerimod.

1. A S1P₁ receptor modulator, or a pharmaceutically acceptable salt thereof, for use in the prevention or treatment of a type 1 interferon mediated disease in a subject, wherein the subject has a high type 1 interferon (IFN-1) gene signature score.

2. A S1P₁ receptor modulator, or a pharmaceutically acceptable salt thereof, for use according to claim 1, wherein the type 1 interferon gene signature score is calculated based on any one of the following groups of type 1 interferon-associated genes:
a. RSAD2, IFI44, IFI44L, and IFI27; or
b. RSAD2, IFI27, IFIT1, and HERC5.

3. A S1P₁ receptor modulator, or a pharmaceutically acceptable salt thereof, for use according to any one of claims 1 or 2, wherein the type 1 interferon gene signature score of the subject is at or above the minimum of the bimodal distribution curve of the type 1 interferon gene signature scores of SLE patients.

4. A S1P₁ receptor modulator, or a pharmaceutically acceptable salt thereof, for use according to any one of claims 1 or 2, wherein the type 1 interferon gene signature score of the subject is above a threshold value, wherein the threshold value of the IFN-1 gene signature score is 2 standard deviations above the arithmetic mean value from at least 100 healthy humans.

5. A S1P₁ receptor modulator, or a pharmaceutically acceptable salt thereof, for use in a method of preventing or treating a subject having a type 1 interferon mediated disease, the method comprising:
a) providing a biological sample from the subject;
b) assaying gene expression of at least three type 1 interferon-associated genes selected from the group consisting of DHX58, EIF2AK2, IRF7, PARP9, SAMD9L, IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6 (especially IFI6, RSAD2, IFI44, IFI44L, IFI27, MX1, IFIT1, HERC5, ISG15, LAMP3, OAS3, OAS1, EPSTI1, IFIT3, LY6E, OAS2, PLSCR1, SIGLEC1, USP18, RTP4, and DNAPTP6, and notably EIF2AK2, IFI6, RSAD2, IFI44, IFI44L, IFI27, MX1, IFIT1, HERC5, and ISG15) in the biological sample;
c) determining a normalised expression value of each assayed gene;
d) calculating a type 1 interferon gene signature score by averaging the normalised expression values; and
e) treating the subject with the S1P₁ receptor modulator when the type 1 interferon gene signature score of the subject is higher than a threshold value.

6. A S1P₁ receptor modulator, or a pharmaceutically acceptable salt thereof, for use according to claim 5, wherein one of the following groups of type 1 interferon-associated genes is selected for assaying gene expression:
a) RSAD2, IFI44, IFI44L, and IFI27; or
b) RSAD2, IFI27, IFIT1, and HERC5.

7. A S1P₁ receptor modulator, or a pharmaceutically acceptable salt thereof, for use in the prevention or treatment of a type 1 interferon mediated disease in a subject, wherein the concentration/level of interferon-gamma (IFN-γ) in the subject is elevated compared to a healthy subject.

8. A S1P₁ receptor modulator, or a pharmaceutically acceptable salt thereof, for use according to any one of claims 1 to 7, wherein the subject has a concentration/level of interferon-alpha (IFN-α) above 0.1 pg/mL and/or of interferon-gamma (IFN-γ) above 9.5 pg/mL.

9. A S1P₁ receptor modulator, or a pharmaceutically acceptable salt thereof, for use according to any one of claims 1 to 8, wherein the S1P₁ receptor modulator is selected from ozanimod, siponimod, ponesimod, cenerimod, etrasimod, mocravimod and amiselimod, or a pharmaceutically acceptable salt thereof.

10. A S1P₁ receptor modulator, or a pharmaceutically acceptable salt thereof, for use according to any one of claims 1 to 8, wherein the S1P₁ receptor modulator is cenerimod.

11. A S1P₁ receptor modulator, or a pharmaceutically acceptable salt thereof, for use according to claim 10, wherein cenerimod is to be administered and/or is administered to a subject for a treatrment period of at least 6 months.

12. A S1P₁ receptor modulator, or a pharmaceutically acceptable salt thereof, for use according to any one of claims 1 to 11, wherein type 1 interferon mediated disease is selected from the group consisting of systemic lupus erythematosus, discoid lupus, lupus nephritis, glomerulonephritis, type I diabetes, inflammatory bowel disease (including Crohn's disease, ulcerative colitis, and Celiac's disease), multiple sclerosis, autoimmune thyroiditis, scleroderma, psoriasis, primary Sjögren's disease, systemic sclerosis, rheumatoid arthritis, transplant rejection, dermatomyositis, polymyositis, idiopathic inflammatory myositis, sarcoidosis, Aicardi-Goutières syndrome, vasculitis, Sting associated vasculopathy with onset in infancy (SAVI) or chronic atypical neutrophilic dermatosis with lipodystrophy and elevanted temperature syndrome (CANDLE).

13. A S1P₁ receptor modulator, or a pharmaceutically acceptable salt thereof, for use according to any one of claims 1 to 11, wherein type 1 interferon mediated disease is systemic lupus erythematosus.

14. A S1P₁ receptor modulator, or a pharmaceutically acceptable salt thereof, for use according to any one of claims 1 to 13, wherein a reduction from baseline in the modified systemic lupus erythematosus disease activity index 2000 (mSLEDAI-2K) is reached after 12 months treatment of the subject with cenerimod.

15. A S1P₁ receptor modulator, or a pharmaceutically acceptable salt thereof, for use according to any one of claims 1 to 14, wherein the subject is a human patient suffering from systemic lupus erythematosus.

16. A S1P₁ receptor modulator, or a pharmaceutically acceptable salt thereof, for use according to any one of claims 1 to 14, wherein the subject is a human patient suffering from systemic lupus erythematosus, and wherein the subject has a mSLEDAI-2K score≥6 and a clinical mSLEDAI-2K score≥4 with at least 2 points for musculoskeletal or mucocutaneous manifestations.
